(19) United States
(12) Patent Application Publication
(10) Pub. No.: US 2023/0250089 A1
(43) Pub. Date: Aug. 10, 2023

(54) MAP4K3 SMALL MOLECULE DRUG INHIBITORS AND METHODS OF USE THEREOF

(71) Applicants: The Regents of the University of California, Oakland, CA (US); Arizona Board of Regents on Behalf of the University of Arizona, Tucson, AZ (US)

(72) Inventors: Albert La Spada, Irvine, CA (US); Daruka Mahadevan, San Antonio, TX (US)

(21) Appl. No.: 18/108,005

(22) Filed: Feb. 9, 2023

Related U.S. Application Data
(60) Provisional application No. 63/308,481, filed on Feb. 9, 2022.

(51) Int. Cl. C07D 403/12; A61K 45/06; C12Q 1/48; C07D 413/12

(52) U.S. Cl. CPC C07D 403/12; A61K 45/06; C12Q 1/485; C07D 413/12

(57) ABSTRACT

The disclosure provides for small molecule inhibitors of the regulatory kinase MAP4K3, and methods of use thereof.

### CROSS REFERENCE TO RELATED APPLICATIONS

This application claims priority under 35 U.S.C. § 119 from Provisional Application Ser. No. 63/308,481 filed Feb. 9, 2022, the disclosure of which is incorporated herein by reference.

### STATEMENT OF GOVERNMENT SUPPORT

This invention was made with Government support under Grant No. R01 AG033082, awarded by the National Institutes of Health. The Government has certain rights in the invention.

### TECHNICAL FIELD

The disclosure provides for small molecule inhibitors of the regulatory kinase MAP4K3, and methods of use thereof.

### BACKGROUND

Mitogen-activated protein kinases (MAPKs) comprise a large family of highly conserved proteins that control a wide range of cellular processes in all eukaryotes. MAP4K3, also known as germinal-center kinase-like kinase, is a member of the Ste20 sub-family of MAPKs, and has been implicated in autoimmune disease via activation of protein kinase C-θ, activation of c-Jun N-terminal kinase (JNK) to promote apoptosis, and the amino acid-stimulated activation of the mechanistic target of rapamycin complex 1 (mTORC1), a multi-protein subunit complex consisting of the catalytic mTOR subunit, mLST8, DEPTOR, the Tti1-Tel2 complex, Raptor, and PRAS409. Studies in mammalian cell lines and in Drosophila have shown that MAP4K3 is absolutely required for activation of mTORC1 in response to amino acids and amino acid levels principally determine the activation status of mTORC1. Furthermore, MAP4K3 is ubiquitously expressed, as MAP4K3 RNA and protein are detected in all human tissues. Thus, MAP4K3 probably has a central role in regulating the metabolic disposition of the cell, but nothing is known as to how MAP4K3 achieves this regulation. Although ATP-site small molecule kinase inhibitors to MAP4K3 are known, these compounds were not further developed due to potential toxicities, stemming from off-target inhibition of other MAPK family members, which is a common problem when targeting the ATP binding pocket.

### SUMMARY

The disclosure provides for small molecule inhibitors of the regulatory kinase MAP4K3, and uses thereof, including for the treatment of various disease and disorders. The MAP4K3 inhibitors were identified by in silico screening and medicinal chemistry. In analyzing the MAP4K3 crystal structure, it was discovered that MAP4K3 exists as a dimer with ordered domain-swapped activation loops, with α-AL and α-EF helices. One monomer's activation loop is bound by a symmetry-mate such that the α-EF helix and loop of one monomer is in a pocket between the α-G helix and the activation loop of its symmetry-mate. It was postulated that a small molecule inhibitor occupying the dimer interface would disrupt the dimer or lock in the dimer, inhibiting enzyme activity.

As such, studies were pursued by using in silico screening of adjacent amino acids to create pharmacophores. The in silico screening utilized SYBYL-X 2.0 software to screen a 2 million compound ZINC database [‘LEADS NOW’] via Lipinski's rules, which yielded ˜2500 hits. Hits were interactively docked into the prospective binding pocket and scored. Compounds were prioritized based upon docking, and 13 compounds were selected for entry into a “important path” of secondary and tertiary screens. This analysis identified four “hits” capable of inhibiting MAP4K3 phosphorylation of PKC-theta by >40%. Furthermore, these four hits robustly promoted TFEB nuclear localization and potently induced autophagy flux.

In a particular embodiment, the disclosure provides a MAP4K3 inhibitor, or a composition or kit comprising thereof, wherein the MAP4K3 inhibitor comprises the structure of Formula I:

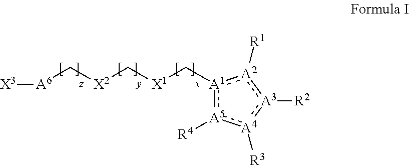

or a pharmaceutically acceptable salt, solvate, or prodrug thereof,

wherein, A¹ is N⁺, C or CR⁵; A² is N, C or CR⁶; A³ is N, C or CR⁷; A⁴ is N, C or CR⁸; A⁵ is N, C or CR⁹; A⁶ is a nitrogen containing optionally substituted heterocycle, wherein the nitrogen containing heterocycle can comprise one or more ring structures, including fused ring structures; R¹-R⁴ are each individually absent or selected from H, D, optionally substituted (C₁-C₁₂)-alkyl, optionally substituted (C₁-C₁₂)-heteroalkyl, optionally substituted (C₂-C₁₂)-alkenyl, optionally substituted (C₂₋₁₂)-heteroalkenyl, optionally substituted (C₂₋₁₂)-alkynyl, optionally substituted (C₂-C₁₂)-heteroalkynyl, optionally substituted (C₃-C₁₂)-cycloalkyl, optionally substituted (C₄-C₁₂)-cycloalkenyl, optionally substituted aryl, optionally substituted heterocycle, halo, hydroxyl, ketone, aldehyde, acyl halide, carbonate, carboxylic acid, ester, ether, amide, amine, imine, azide, cyanate, azo, nitrate, nitrile, nitro, nitroso, thiol, sulfide, disulfide, sulfoxide, sulfone, sulfinic acid, sulfonic acid, thiocyanate, thione, thial, phosphine, phosphonic acid, phosphate, phosphodiester, boronic acid, boronic ester, borinic acid, and borinic ester; R⁵-R⁹ are each individually selected from H, D, optionally substituted (C₁-C₁₂)-alkyl, optionally substituted (C₁-C₁₂)-heteroalkyl, optionally substituted (C₂-C₁₂)-alkenyl, optionally substituted (C₂₋₁₂)-heteroalkenyl, optionally substituted (C₂₋₁₂)-alkynyl, optionally substituted (C₂-C₁₂)-heteroalkynyl, optionally substituted (C₃-C₁₂)-cycloalkyl, optionally substituted (C₄-C₁₂)-cycloalkenyl, optionally substituted aryl, optionally substituted heterocycle, halo, hydroxyl, ketone, aldehyde, acyl halide, carbonate, carboxylic acid, ester, ether, amide, amine, imine, azide, cyanate, azo, nitrate, nitrile, nitro, nitroso, thiol, sulfide, disulfide, sulfoxide, sulfone, sulfinic acid, sulfonic acid, thiocyanate, thione, thial, phosphine, phosphonic acid, phosphate, phosphodiester, boronic acid, boronic ester, borinic acid, and borinic ester; R¹⁰-R¹⁴ are each individually selected from H, D, halo, and an optionally substituted (C₁-C₆)-alkyl; X¹ is S, O, CR¹⁰R¹¹ or NR¹²; X² is NR¹³,

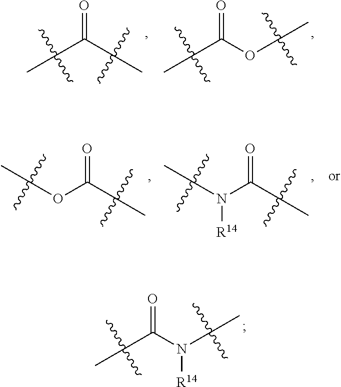

X³ is selected from H, D, halo, hydroxyl, amine, optionally substituted (C₁-C₆)-alkyl, azido, nitrile, and a (C₁-C₆)-alkylammonium group; x is an integer selected from 0, 1, 2, or 3; y is an integer selected from 0, 1, 2 or 3; z is an integer selected from 0, 1, 2, or 3; wherein, at least 2 of A¹-A⁵ comprise nitrogen containing groups, wherein, there is only one double bond located between one of the following recited groups: A¹ and A⁵, and A¹ and A²; and wherein, there is only one double bond located between one of the following recited groups: A² and A³, A³ and A⁴, and A⁴ and A⁵. In a further embodiment, the MAP4K3 inhibitor of Formula I does not have a structure selected from

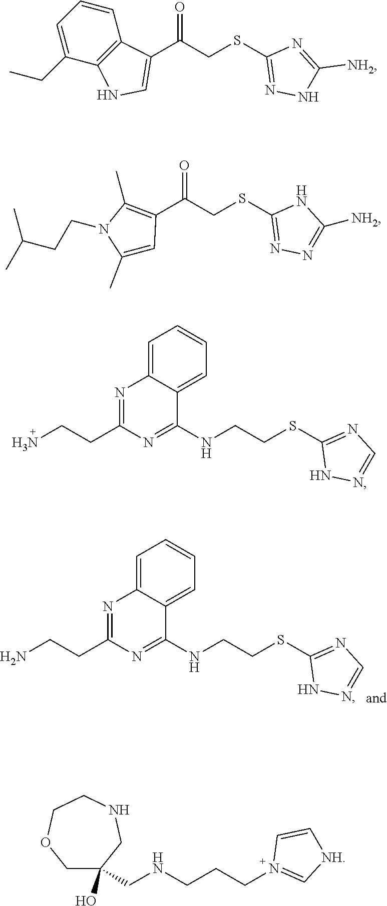

In another embodiment, A6 is selected from:

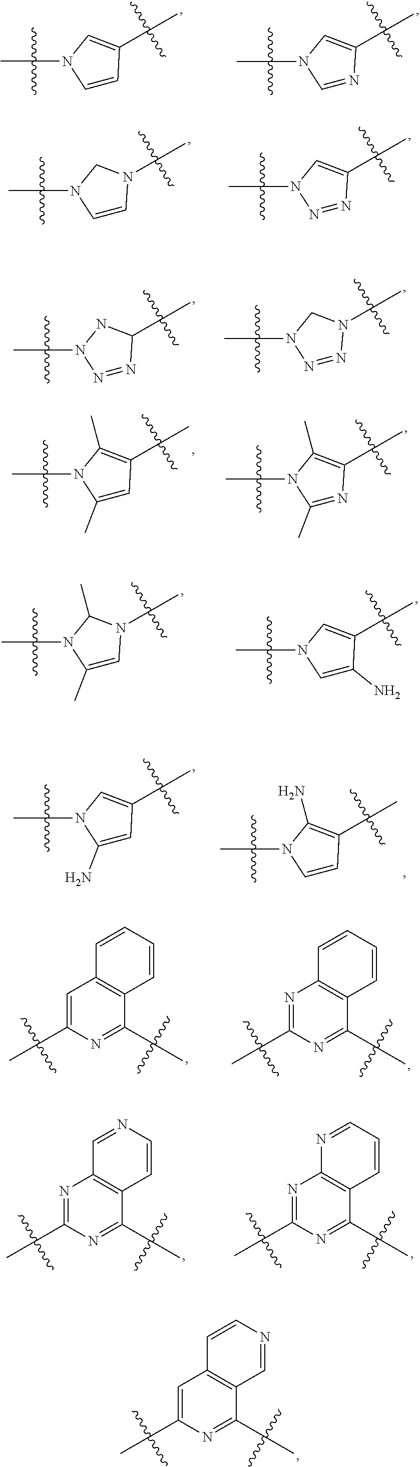

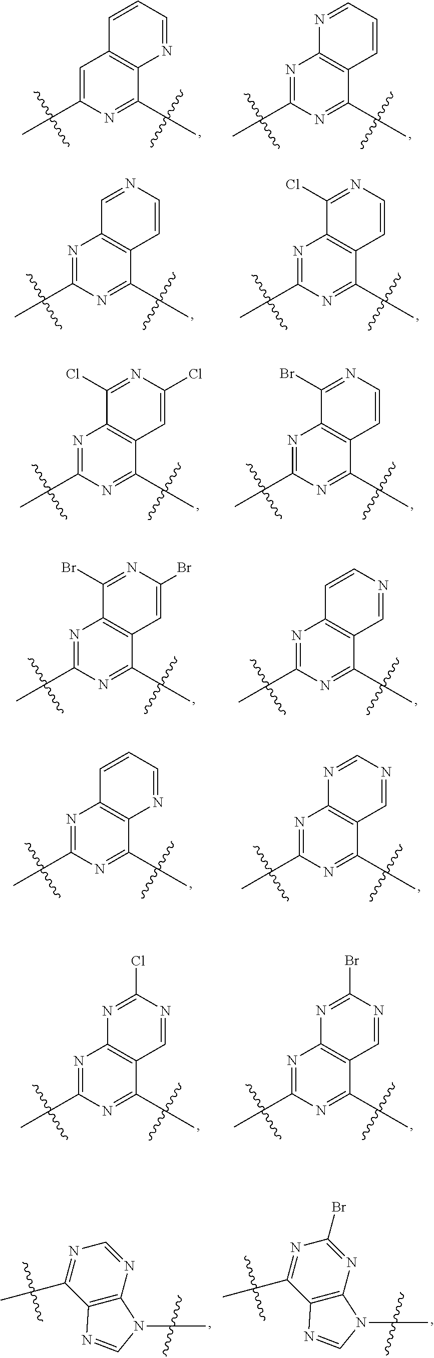

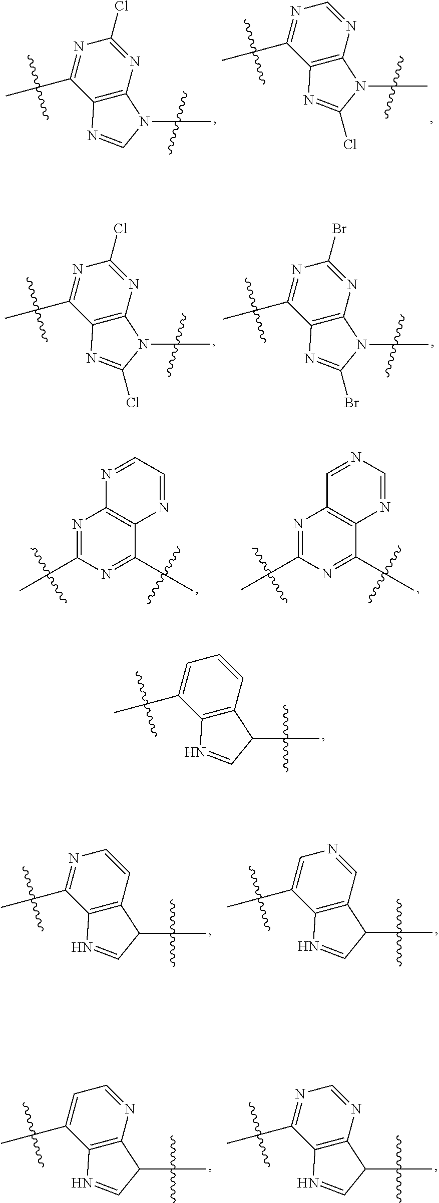

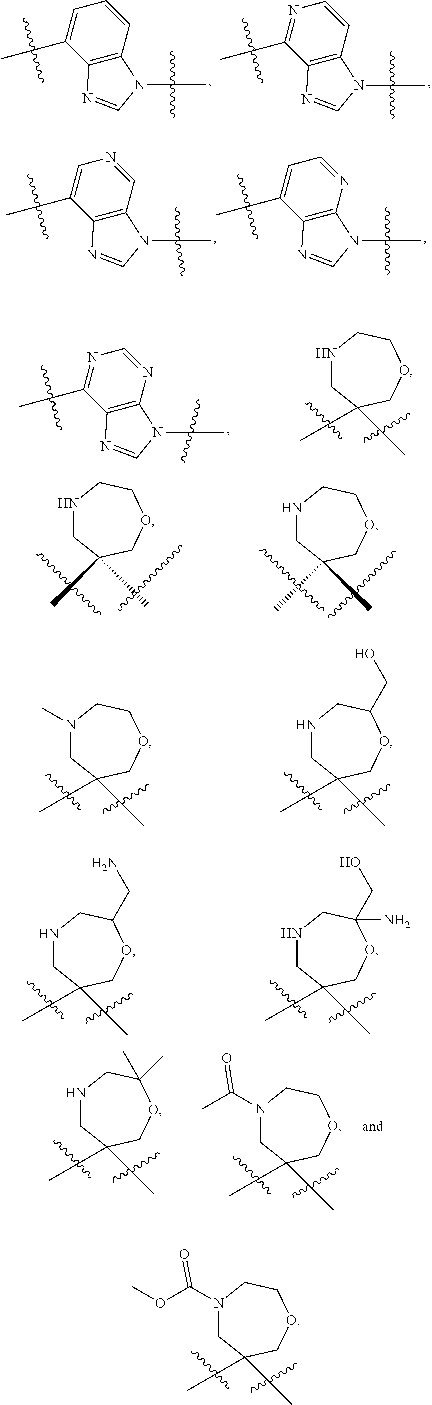

In yet another embodiment, the MAP4K3 inhibitor comprises the structure of Formula I(a):

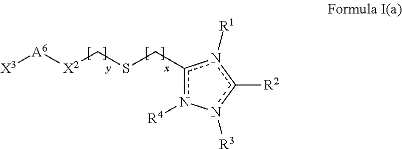

or a pharmaceutically acceptable salt, solvate, or prodrug thereof,
wherein, A⁶ is a nitrogen containing optionally substituted heterocycle, wherein the nitrogen containing heterocycle can comprise one or more ring structures, including fused ring structures; R¹-R⁴ are each individually absent or selected from H, D, optionally substituted (C₁-C₆)-alkyl, optionally substituted (C₁-C₆)-heteroalkyl, optionally substituted (C₂-C₆)-alkenyl, optionally substituted (C₂₋₆)-heteroalkenyl, optionally substituted (C₂₋₆)-alkynyl, optionally substituted (C₂-C₆)-heteroalkynyl, optionally substituted (C₃-C₈)-cycloalkyl, optionally substituted (C₄-C₈)-cycloalkenyl, optionally substituted aryl, optionally substituted heterocycle, halo, hydroxyl, ketone, aldehyde, acyl halide, carbonate, carboxylic acid, ester, ether, amide, amine, imine, azide, cyanate, azo, nitrate, nitrile, nitro, and nitroso, thiol, sulfide, disulfide, sulfoxide, sulfone, sulfinic acid, sulfonic acid, thiocyanate, thione, thial, phosphine, phosphonic acid, phosphate, phosphodiester, boronic acid, boronic ester, borinic acid, and borinic ester; R¹³-R¹⁴ are each individually selected from H, D, halo, and an optionally substituted (C₁-C₆)-alkyl; X² is an NR¹³,

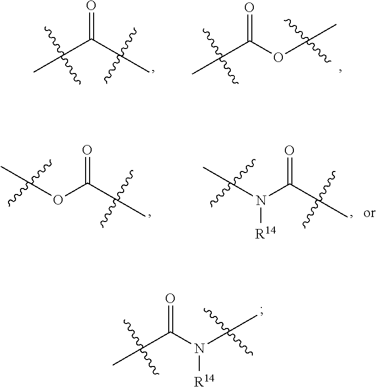

X³ is selected from H, D, halo, hydroxyl, amine, optionally substituted (C₁-C₆)-alkyl, azido, and nitrile; x is an integer selected from 0 or 1; y is an integer selected from 1, 2, or 3; and z is an integer selected from 0 or 1. In a further embodiment, the MAP4K3 inhibitor of Formula (I) does not have a structure selected from:

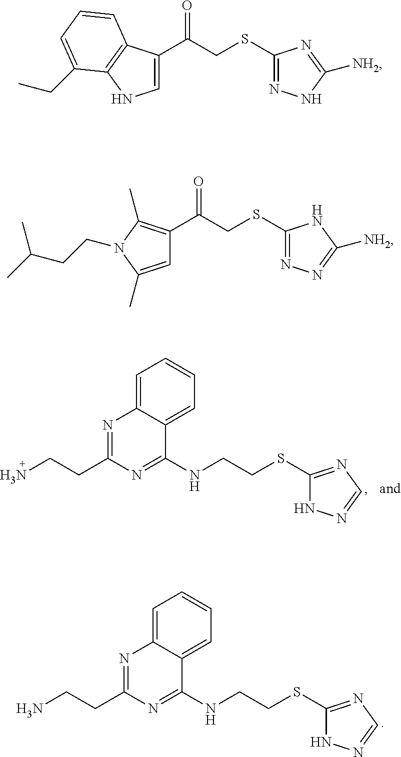 [SMILES: CCc1cccc2c(C(=O)CSc3n[nH]c(N)n3)c[nH]c12 ; Cc1cc(C(=O)CSc2nnc(N)[nH]2)c(C)n1CCC(C)C ; NCCc1nc(NCCSc2ncn[nH]2)c2ccccc2n1 ; NCCc1nc(NCCSc2ncn[nH]2)c2ccccc2n1]

In yet a further embodiment, A⁶ is selected from:

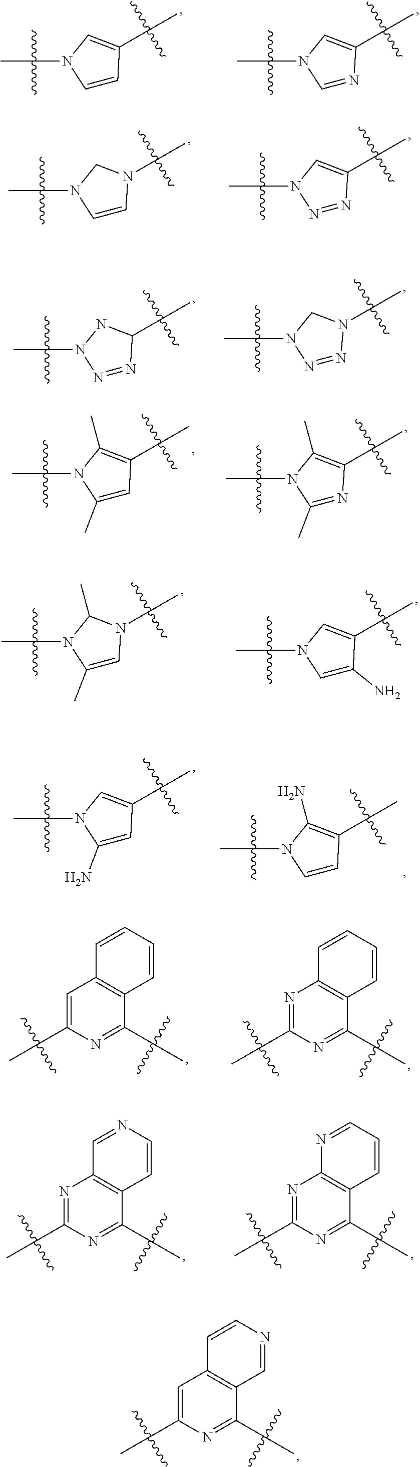

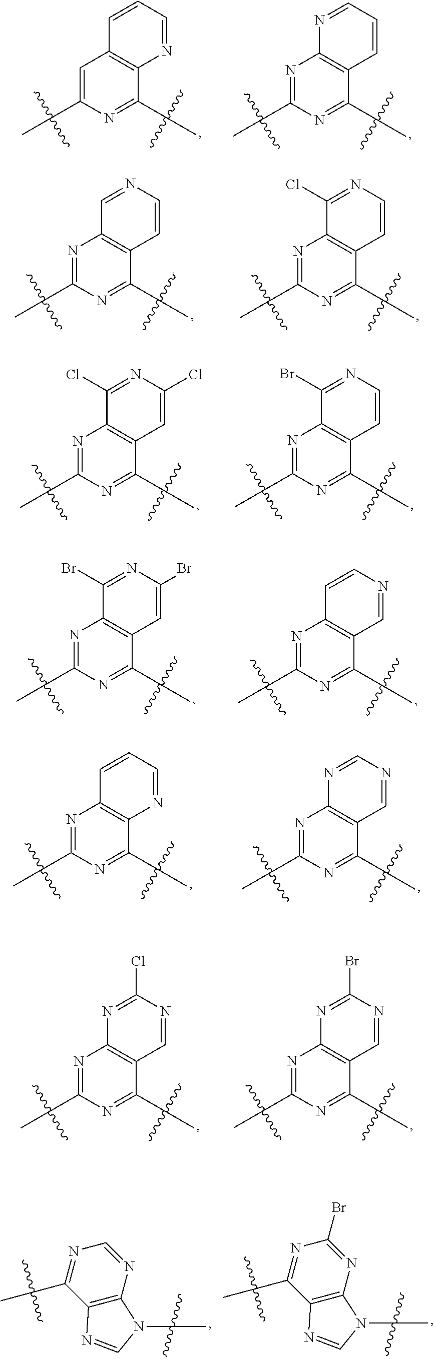

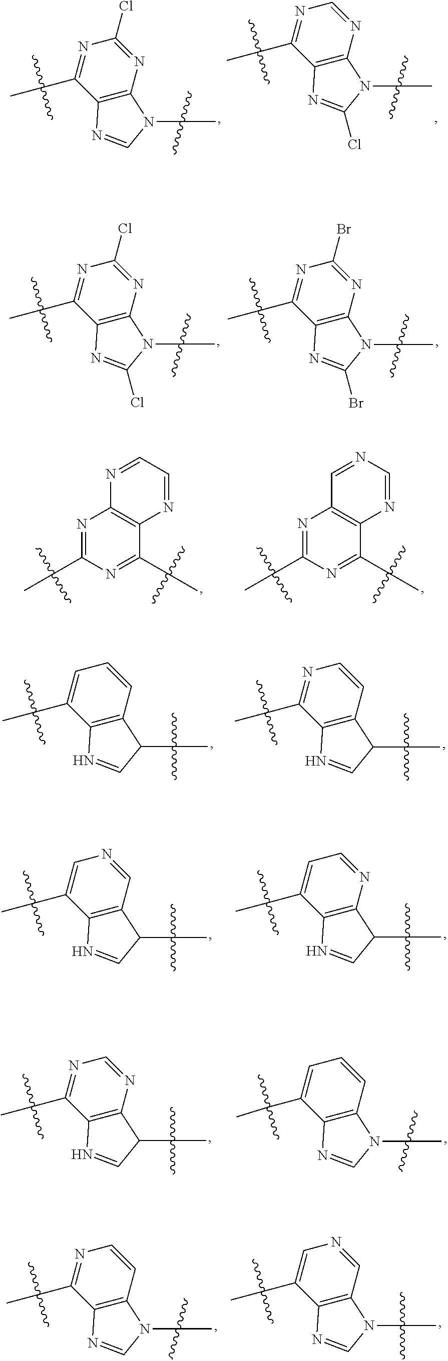

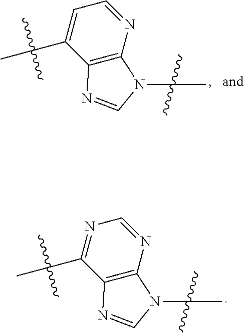 [SMILES: *c1ccnc2c1ncn2* ; *c1ncnc2c1ncn2*]

In yet a further embodiment, the MAP4K3 inhibitor comprises the structure of Formula I(b):

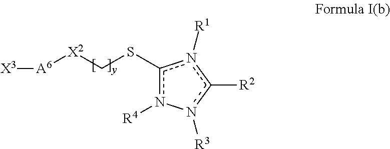

or a pharmaceutically acceptable salt, solvate, or prodrug thereof,
wherein, A⁶ is selected from

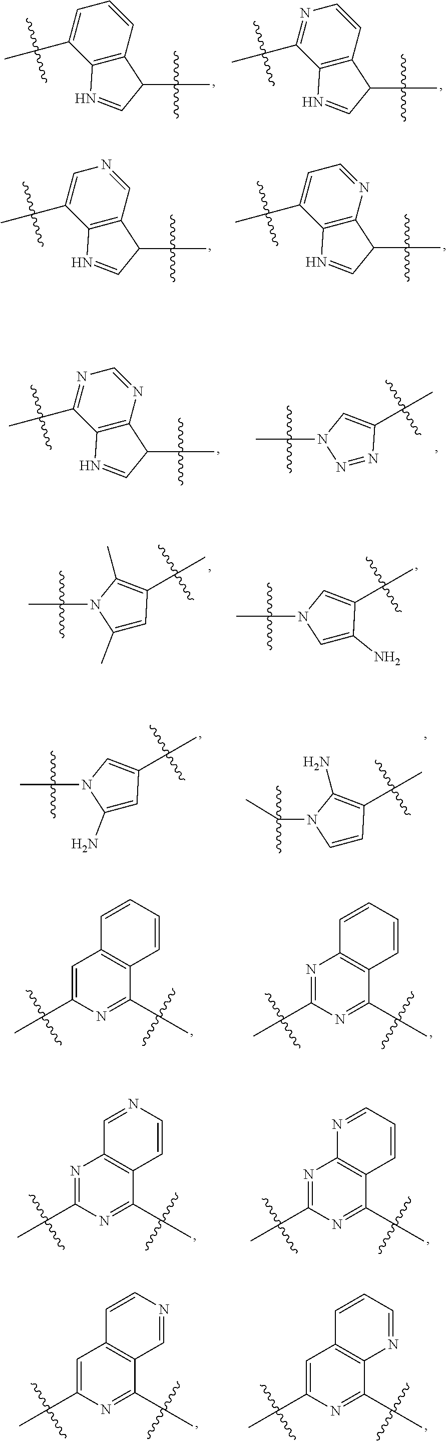

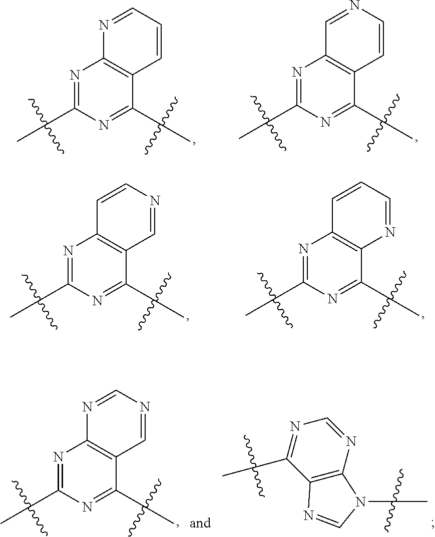 [SMILES: *c1nc(*)c2cccnc2n1 ; *c1nc(*)c2ccncc2n1 ; *c1nc(*)c2cnccc2n1 ; *c1nc(*)c2ncccc2n1 ; *c1nc(*)c2cncnc2n1 ; *c1ncnc2c1ncn2*]

R¹-R⁴ are each individually absent or selected from H, D, optionally substituted (C₁-C₆)-alkyl, optionally substituted (C₁-C₆)-heteroalkyl, optionally substituted (C₂-C₆)-alkenyl, optionally substituted (C₂₋₆)-heteroalkenyl, optionally substituted (C₂₋₆)-alkynyl, optionally substituted (C₂-C₆)-heteroalkynyl, optionally substituted (C₃-C₈)-cycloalkyl, optionally substituted (C₄-C₈)-cycloalkenyl, optionally substituted aryl, optionally substituted heterocycle, halo, hydroxyl, ketone, aldehyde, acyl halide, carbonate, carboxylic acid, ester, ether, amide, amine, imine, azide, cyanate, azo, nitrate, nitrile, nitro, and nitroso, thiol, sulfide, disulfide, sulfoxide, sulfone, sulfinic acid, sulfonic acid, thiocyanate, thione, thial, phosphine, phosphonic acid, phosphate, phosphodiester, boronic acid, boronic ester, borinic acid, and borinic ester; R¹³ is selected from H, D, halo, and an optionally substituted (C₁-C₆)-alkyl; X² is NR¹³ or

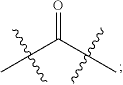 [SMILES: *C(*)=O]

X³ is an optionally substituted (C₁-C₆)-alkyl group; and y is an integer selected from 1, 2, or 3. In a further embodiment, the MAP4K3 inhibitor of Formula I(b) does not have a structure selected from:

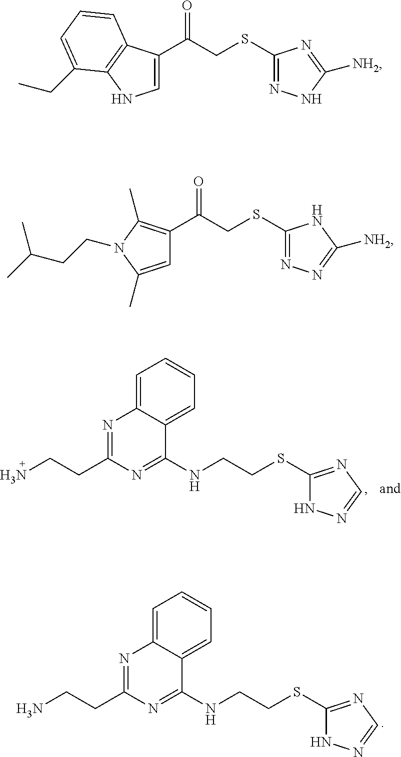 [SMILES: CCc1cccc2c(C(=O)CSc3n[nH]c(N)n3)c[nH]c12 ; Cc1cc(C(=O)CSc2nnc(N)[nH]2)c(C)n1CCC(C)C ; NCCc1nc(NCCSc2ncn[nH]2)c2ccccc2n1 ; NCCc1nc(NCCSc2ncn[nH]2)c2ccccc2n1]

In a certain embodiment, the MAP4K3 inhibitor comprises the structure of Formula I(c):

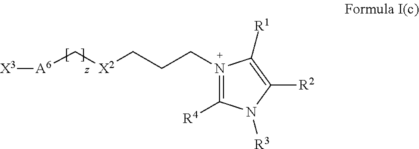

or a pharmaceutically acceptable salt, solvate, or prodrug thereof,

wherein, A⁶ is selected from

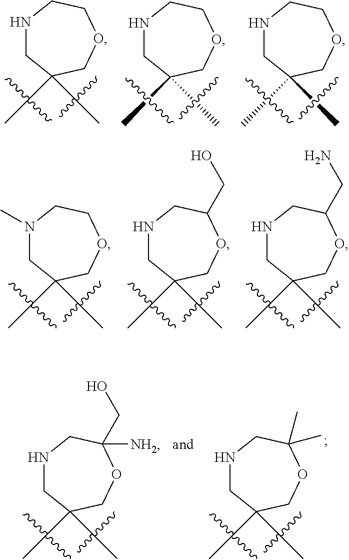

R¹³-R¹⁴ are each individually selected from H and an optionally substituted (C₁-C₆)-alkyl; X² is selected from NR¹³,

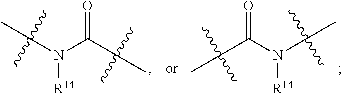

X³ is selected from H, D, halo, hydroxyl, amine, azido, and a nitrile; and z is an integer selected from 1, 2, or 3. In a further embodiment, the MAP4k3 inhibitor of Formula I(c) does not have a structure of:

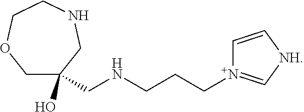 [SMILES: O[C@]1(CNCCC[n+]2cc[nH]c2)CNCCOC1]

In yet a further embodiment, the MAP4K3 inhibitor has a structure selected from (a), (b), (c), (d) and (e):

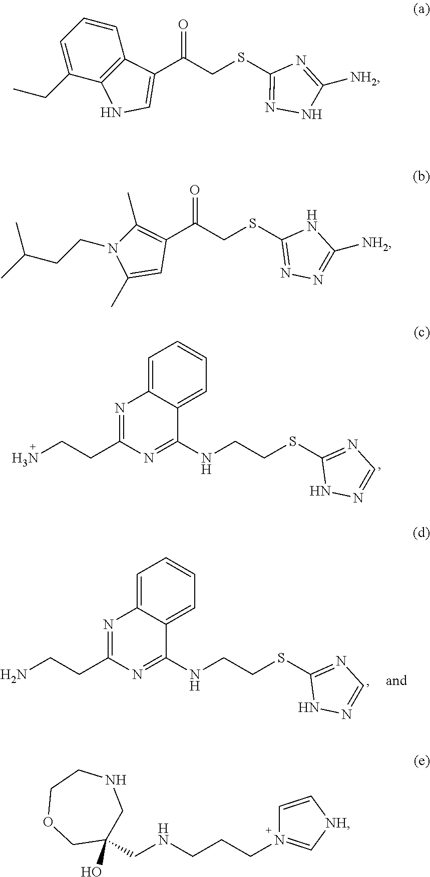

or a pharmaceutically acceptable salt, solvate, or prodrug thereof. In a further embodiment, the MAP4K3 inhibitor inhibits MAP4K3 phosphorylation of PKCθThr538/total PKCθ by at least 40%.

In a particular embodiment, the disclosure also provides a pharmaceutical composition comprising a MAP4K3 inhibitor disclosed herein and a pharmaceutically acceptable excipient, diluent and/or carrier. In a further embodiment, the pharmaceutical composition is formulated for oral, parenteral, or topical delivery.

In a certain embodiment, the disclosure further provides a method of treating a MAP4K3-mediated disorder in a subject, comprising administering to the subject in need thereof a therapeutically effective amount of the MAP4K3 inhibitor disclosed herein, or a pharmaceutical composition comprising a MAP4K3 inhibitor disclosed herein. In another embodiment, the MAP4K3-mediated disorder is a disorder associated with overexpression of MAP4K3, mis-regulation of MAP4K3, abnormal levels of MAP4K3, and/or abnormal activity of MAP4K3. In yet another embodiment, the MAP4K3-mediated disorder is a cancer, neurological disease, autoimmune disorder, or aging. In yet a further embodiment, the cancer is selected from metastatic cancer, human renal cancer, lung cancer, liver cancer, pancreatic cancer, prostate cancer, endometrial cancer, melanoma, glioblastoma, and thyroid cancer. In another embodiment, the neurological disease is selected from tuberous sclerosis, epilepsy, Fragile X syndrome, Down syndrome, Rett syndrome, Alzheimer's disease, Parkinson's disease, and Huntington's disease. In yet another embodiment, the autoimmune disorder is selected from collagen induced arthritis (CIA), experimental autoimmune encephalomyelitis (EAE), inflammatory bowel disease (IBD), systemic lupus erythematosus (SLE), rheumatoid arthritis (RA), multiple sclerosis (MS), Type 1 diabetes mellitus, Guillain-Barre syndrome, psoriasis, chronic inflammatory demyelinating polyneuropathy, Graves' disease, Hashimoto's thyroiditis, myasthenia gravis, vasculitis, Sjögren syndrome, Addison disease, celiac disease, dermatomyositis, and adult-onset Still's disease (AOSD). In a further embodiment, the MAP4K3 inhibitor is administered in combination with one or more additional therapeutics or agents.

In a particular embodiment, the disclosure provides a screening method to identify MAP4K3 inhibitors, comprising: identifying compounds in an in vitro kinase assay which inhibit the phosphorylation of PKCθThr538/total PKCθ by at least 40%. In another embodiment, the method further comprises one or more of the followings steps for the compounds that are identified to inhibit the phosphorylation of PKCθThr538/total PKCθ by at least 40%: identifying compounds that provide for at least 50% of TFEB in nuclei; and/or identifying compounds that provide for at least 20% increase in LC3-II flux/LC3-I normalized to β-actin+/−bafilomycin; and/or identifying compounds that provide for at least a 2.0-fold increase in the number of autolysosomes.

In a particular embodiment, the disclosure provides a kit comprising a MAP4K3 inhibitor disclosed herein.

In a certain embodiment, the disclosure also provides a MAP4K3 inhibitor, a method of using the MAP4K3 inhibitor, or a kit comprising the MAP4K3 inhibitor, as substantially described herein.

The details of one or more embodiments of the disclosure are set forth in the accompanying drawings and the description below. Other features, objects, and advantages will be apparent from the description and drawings, and from the claims.

### DESCRIPTION OF DRAWINGS

FIG. 1 presents a three-dimensional structure model depicting the dimerization of two MAP4K3 kinase domains. The active site of MAP4K3 kinase domain is indicated.

FIG. 2 provides an exemplary flowchart to evaluate candidate MAPK43 inhibitors. Candidate MAP4K3 inhibitors will undergo an initial secondary screen to confirm kinase inhibition toward an established substrate, and then the “hits” will be tested for their ability to promote TFEB nuclear localization and to activate autophagy, in order to assess biological relevance in tertiary and quaternary validation assays.

FIG. 3 presents exemplary structures of MAP4K3 inhibitors identified using the screening method described in FIG. 2.

FIG. 4A-C provides for the biological validation of candidate MAPK43 inhibitors. (A) HEK293A cells transfected with TFEB-FLAG were cultured under complete media (CM) and treated with each of 3 compounds at the μM concentrations, as indicated. WT=negative ctrl.; MAP4K3 KO cells=positive ctrl. Compound #2189 and #4479 promoted TFEB nuclear localization. (B) LC3 immunoblotting of HEK293 cells cultured in CM+/−bafilomycin and treated with compound #3396 at the concentrations indicated to measure LC3-II flux. AA starvation=positive ctrl. (C) Number of red puncta (autolysosomes) counted in HEK293 cells cultured in CM, transfected with GFP-LC3-mCherry, and treated with 2 compounds as indicated. AA starvation=positive ctrl. *P<0.05, **P<0.01, ***P<0.001; ANOVA with post-hoc Tukey test. Error bars=s.e.m.

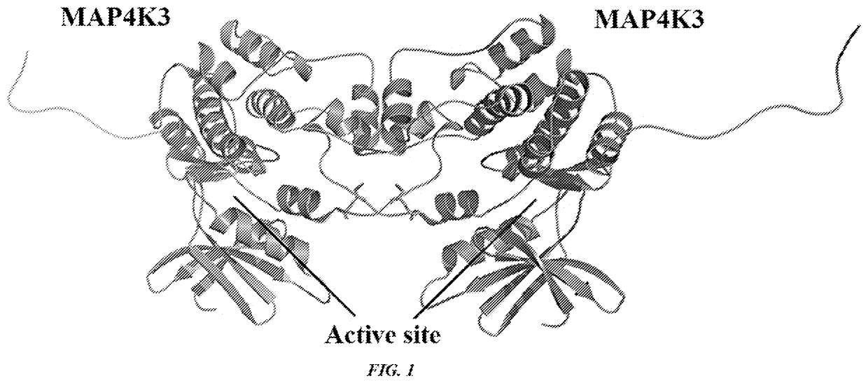

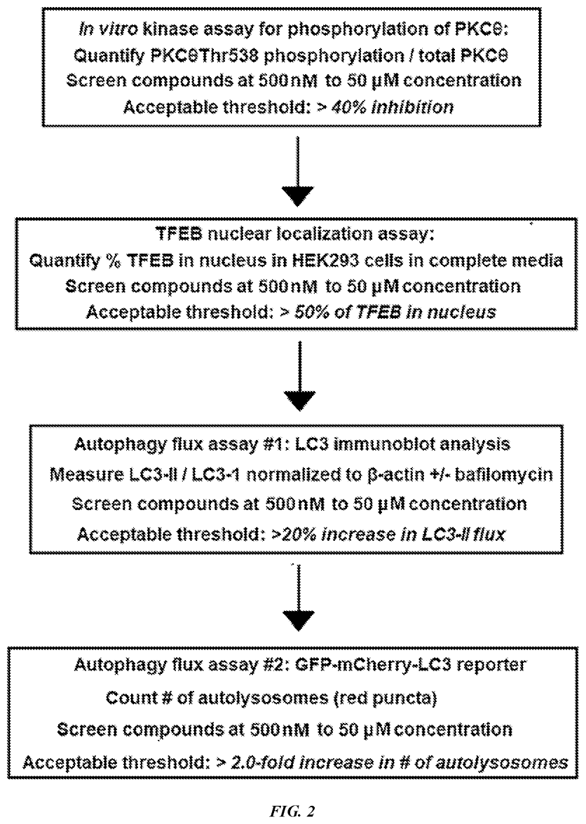

### DETAILED DESCRIPTION

As used herein and in the appended claims, the singular forms “a,” “an,” and “the” include plural referents unless the context clearly dictates otherwise. Thus, for example, reference to “a MAP4K3 inhibitor” includes a plurality of such inhibitors and reference to “the amino acid” includes reference to one or more amino acids and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

The term “alkenyl”, refers to an organic group that is comprised of carbon and hydrogen atoms that contains at least one double covalent bond between two carbons. Typically, an “alkenyl” as used in this disclosure, refers to organic group that contains 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. While a C₂-alkenyl can form a double bond to a carbon of a parent chain, an alkenyl group of three or more carbons can contain more than one double bond. It certain instances the alkenyl group will be conjugated, in other cases an alkenyl group will not be conjugated, and yet other cases the alkenyl group may have stretches of conjugation and stretches of nonconjugation. Additionally, if there is more than 2 carbon, the carbons may be connected in a linear manner, or alternatively if there are more than 3 carbons then the carbons may also be linked in a branched fashion so that the parent chain contains one or more secondary, tertiary, or quaternary carbons. An alkenyl may be substituted or unsubstituted, unless stated otherwise.

The term “alkyl”, refers to an organic group that is comprised of carbon and hydrogen atoms that contains single covalent bonds between carbons. Typically, an “alkyl” as used in this disclosure, refers to an organic group that contains 1, 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. Where if there is more than 1 carbon, the carbons may be connected in a linear manner, or alternatively if there are more than 2 carbons then the carbons may also be linked in a branched fashion so that the parent chain contains one or more secondary, tertiary, or quaternary carbons. An alkyl may be substituted or unsubstituted, unless stated otherwise.

The term “alkynyl”, refers to an organic group that is comprised of carbon and hydrogen atoms that contains a triple covalent bond between two carbons. Typically, an “alkynyl” as used in this disclosure, refers to organic group that contains that contains 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. While a C₂-alkynyl can form a triple bond to a carbon of a parent chain, an alkynyl group of three or more carbons can contain more than one triple bond. Where if there is more than 3 carbon, the carbons may be connected in a linear manner, or alternatively if there are more than 4 carbons then the carbons may also be linked in a branched fashion so that the parent chain contains one or more secondary, tertiary, or quaternary carbons. An alkynyl may be substituted or unsubstituted, unless stated otherwise.

The term “aryl”, as used in this disclosure, refers to a conjugated planar ring system with delocalized pi electron clouds that contain only carbon as ring atoms. An “aryl” for the purposes of this disclosure encompasses from 1 to 4 aryl rings wherein when the aryl is greater than 1 ring the aryl rings are joined so that they are linked, fused, or a combination thereof. An aryl may be substituted or unsubstituted, or in the case of more than one aryl ring, one or more rings may be unsubstituted, one or more rings may be substituted, or a combination thereof.

For purposes of the disclosure the term “cancer” will be used to encompass cell proliferative disorders, neoplasms, precancerous cell disorders and cancers, unless specifically delineated otherwise. Thus, a “cancer” refers to any cell that undergoes aberrant cell proliferation that can lead to metastasis or tumor growth. Exemplary cancers include but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, including triple negative breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, Merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, soft tissue sarcoma, uterine cancer, uterine sarcoma, skin cancer (non-melanoma), skin cancer (melanoma), papillomas, actinic keratosis and keratoacanthomas, merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

The term generally represented by the notation “Cₓ-C_{y}” (where x and y are whole integers and y>x) prior to a functional group, e.g., “C₁-C₁₂ alkyl” refers to a number range of carbon atoms. For the purposes of this disclosure any range specified by “Cₓ-C_{y}” (where x and y are whole integers and y>x) is not exclusive to the expressed range, but is inclusive of all possible ranges that include and fall within the range specified by “Cₓ-C_{y}” (where x and y are whole integers and y>x). For example, the term “C₁-C₄” provides express support for a range of 1 to 4 carbon atoms, but further provides implicit support for ranges encompassed by 1 to 4 carbon atoms, such as 1 to 2 carbon atoms, 1 to 3 carbon atoms, 2 to 3 carbon atoms, 2 to 4 carbon atoms, and 3 to 4 carbon atoms.

The term “cycloalkenyl”, as used in this disclosure, refers to an alkene that contains at least 4 carbon atoms but no more than 12 carbon atoms connected so that it forms a ring. A “cycloalkenyl” for the purposes of this disclosure encompasses from 1 to 4 cycloalkenyl rings, wherein when the cycloalkenyl is greater than 1 ring, then the cycloalkenyl rings are joined so that they are linked, fused, or a combination thereof. A cycloalkenyl may be substituted or unsubstituted, or in the case of more than one cycloalkenyl ring, one or more rings may be unsubstituted, one or more rings may be substituted, or a combination thereof.

The term “cycloalkyl”, as used in this disclosure, refers to an alkyl that contains at least 3 carbon atoms but no more than 12 carbon atoms connected so that it forms a ring. A “cycloalkyl” for the purposes of this disclosure encompasses from 1 to 4 cycloalkyl rings, wherein when the cycloalkyl is greater than 1 ring, then the cycloalkyl rings are joined so that they are linked, fused, or a combination thereof. A cycloalkyl may be substituted or unsubstituted, or in the case of more than one cycloalkyl ring, one or more rings may be unsubstituted, one or more rings may be substituted, or a combination thereof.

The term “hetero-” when used as a prefix, such as, hetero-alkyl, hetero-alkenyl, hetero-alkynyl, or hetero-hydrocarbon, for the purpose of this disclosure refers to the specified hydrocarbon having one or more carbon atoms replaced by non-carbon atoms as part of the parent chain. Examples of such non-carbon atoms include, but are not limited to, N, O, S, Si, Al, B, and P. If there is more than one non-carbon atom in the hetero-based parent chain then this atom may be the same element or may be a combination of different elements, such as N and O. In a particular embodiment, a “hetero”-hydrocarbon (e.g., alkyl, alkenyl, alkynyl) refers to a hydrocarbon that has from 1 to 3 C, N and/or S atoms as part of the parent chain.

The term “disorder” as used herein is intended to be generally synonymous, and is used interchangeably with, the terms “disease,” “syndrome,” and “condition” (as in medical condition), in that all reflect an abnormal condition of the human or animal body or of one of its parts that impairs normal functioning, is typically manifested by distinguishing signs and symptoms.

The term “heterocycle,” as used herein, refers to ring structures that contain at least 1 noncarbon ring atom. A “heterocycle” for the purposes of this disclosure encompasses from 1 to 4 heterocycle rings, wherein when the heterocycle is greater than 1 ring the heterocycle rings are joined so that they are linked, fused, or a combination thereof. A heterocycle may be aromatic or nonaromatic, or in the case of more than one heterocycle ring, one or more rings may be nonaromatic, one or more rings may be aromatic, or a combination thereof. A heterocycle may be substituted or unsubstituted, or in the case of more than one heterocycle ring one or more rings may be unsubstituted, one or more rings may be substituted, or a combination thereof. Typically, the noncarbon ring atom is N, O, S, Si, Al, B, or P. In the case where there is more than one noncarbon ring atom, these noncarbon ring atoms can either be the same element, or combination of different elements, such as N and O. Examples of heterocycles include, but are not limited to: a monocyclic heterocycle such as, aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide; and polycyclic heterocycles such as, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine. In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

The terms “heterocyclic group”, “heterocyclic moiety”, “heterocyclic”, or “heterocyclo” used alone or as a suffix or prefix, refers to a heterocycle that has had one or more hydrogens removed therefrom.

The term “hydrocarbons” refers to groups of atoms that contain only carbon and hydrogen. Examples of hydrocarbons that can be used in this disclosure include, but are not limited to, alkanes, alkenes, alkynes, arenes, and benzyls.

The term “non-release controlling excipient” as used herein, refers to an excipient whose primary function do not include modifying the duration or place of release of the active substance from a dosage form as compared with a conventional immediate release dosage form.

The term “optionally substituted” refers to a functional group, typically a hydrocarbon or heterocycle, where one or more hydrogen atoms may be replaced with a substituent. Accordingly, “optionally substituted” refers to a functional group that is substituted, in that one or more hydrogen atoms are replaced with a substituent, or unsubstituted, in that the hydrogen atoms are not replaced with a substituent. For example, an optionally substituted hydrocarbon group refers to an unsubstituted hydrocarbon group or a substituted hydrocarbon group. In a particular embodiment, “optionally substituted” refers to substituents that replace one or more hydrogen atoms which are selected from alkyl, heteroalkyl, alkenyl, heteroalkenyl, alkynyl, heteroalkynyl, cycloalkyl, cycloalkenyl, aryl, heterocycle, halo, hydroxyl, ketone, aldehyde, acyl halide, carbonate, carboxylic acid, ester, ether, amide, amine, imine, azide, cyanate, azo, nitrate, nitrile, nitro, nitroso, thiol, sulfide, disulfide, sulfoxide, sulfone, sulfinic acid, sulfonic acid, thiocyanate, thione, thial, phosphine, phosphonic acid, phosphate, phosphodiester, boronic acid, boronic ester, borinic acid, and/or borinic ester. It should be further noted, that in regards to groups like alkyl, heteroalkyl, alkenyl, heteroalkenyl, alkynyl, heteroalkynyl, cycloalkyl, cycloalkenyl, and aryl, these groups may be unsubstituted or substituted.

The term “pharmaceutically acceptable carrier,” “pharmaceutically acceptable excipient,” “physiologically acceptable carrier,” or “physiologically acceptable excipient” as used herein, refers to a pharmaceutically-acceptable material, composition, or vehicle, such as a liquid or solid filler, diluent, excipient, solvent, or encapsulating material. Each component should be “pharmaceutically acceptable” in the sense of being compatible with the other ingredients of a pharmaceutical formulation. It should also be suitable for use in contact with the tissue or organ of humans and animals without excessive toxicity, irritation, allergic response, immunogenicity, or other problems or complications, commensurate with a reasonable benefit/risk ratio. Examples of “pharmaceutically acceptable carriers” and “pharmaceutically acceptable excipients” can be found in the following, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004.

The term “release controlling excipient” as used herein, refers to an excipient whose primary function is to modify the duration or place of release of the active substance from a dosage form as compared with a conventional immediate release dosage form.

The term “therapeutically acceptable” refers to those compounds (or salts, prodrugs, tautomers, zwitterionic forms, etc.) which are suitable for use in contact with the tissues of patients without excessive toxicity, irritation, allergic response, immunogenicity, are commensurate with a reasonable benefit/risk ratio, and are effective for their intended use.

The terms “treat”, “treating” and “treatment”, as used herein, refers to ameliorating symptoms associated with a disease or disorder (e.g., multiple sclerosis), including preventing or delaying the onset of the disease or disorder symptoms, and/or lessening the severity or frequency of symptoms of the disease or disorder.

The term “subject” as used herein, refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein. For example, a mammalian subject can refer to a human patient.

The term “substituent” refers to an atom or group of atoms substituted in place of a hydrogen atom. For purposes of this invention, a substituent would include deuterium atoms.

The term “substituted” with respect to hydrocarbons, heterocycles, and the like, refers to structures wherein the parent chain contains one or more substituents.

The term “unsubstituted” with respect to hydrocarbons, heterocycles, and the like, refers to structures wherein the parent chain contains no substituents.

The MAP4K (MAP kinase kinase kinase kinase or MAPKKKK) family kinases are serine/threonine kinases, which belong to the mammalian Ste20-like kinase family. Overexpression of MAP4K family kinases specifically induces activation of JNK, but not p38 or ERK, in mammalian cells. MAP4K3 is a member of the MAP4K family. Studies in mammalian cell lines and in Drosophila have shown that MAP4K3 is absolutely required for activation of mTORC1 in response to amino acids and amino acid levels principally determine the activation status of mTORC1. Furthermore, MAP4K3 is ubiquitously expressed, as MAP4K3 RNA and protein are detected in all human tissues.

MAP4K3 is an amino acid-dependent regulator of autophagy through its phosphorylation of transcription factor EB (TFEB), a transcriptional activator of autophagy, and through amino acid starvation-dependent lysosomal localization of MAP4K3. MAP4K3 physically interacts with TFEB and MAP4K3 inhibition is sufficient for TFEB nuclear localization, target gene transactivation, and autophagy, even when mTORC1 is activated. Moreover, MAP4K3 serine 3 phosphorylation of TFEB is required for TFEB interaction with mTORC1-Rag GTPase-Regulator complex and TFEB cytosolic sequestration. The results uncover a role for MAP4K3 in the control of autophagy and reveal MAP4K3 as a central node in nutrient-sensing regulation.

MAP4K3 overexpression contributes to autoimmune responses. MAP4K3-deficient mice display decreased disease scores in autoimmune disease models such as collagen induced arthritis (CIA) and experimental autoimmune encephalomyelitis (EAE) (Chuang et al., Nat Immunol. 12(11):1113-8 (2011); Chuang et al., FASEB J 33(10):11469-11480 (2019)). Consistently, the percentage of MAP4K3-overexpressing T cells is enhanced in the peripheral blood from patients with human autoimmune diseases, including systemic lupus erythematosus (SLE), rheumatoid arthritis (RA), and adult-onset Still's disease (AOSD) (Chuang et al., Nat Immunol. 12(11):1113-8 (2011); Chuang et al., FASEB J 33(10):11469-11480 (2019); Chen et al., BMC Med. 10:84 (2012); and Chen et al., Arthritis Rheum 65:2573-82 (2013)). The MAP4K3-overexpressing T cell population is correlated with disease severity of autoimmune disease patients; therefore, MAP4K3-overexpressing T cell is a biomarker for autoimmune diseases (Chaung et al., Journal of Biomedical Sciences 26:82 (2019)). Accordingly, inhibition of MAP4K3 may be useful for treating autoimmune and neurological disease patients.

MAP4K3 overexpression occurs in cancer tissues of human non-small cell lung carcinoma (NSCLC), hepatocellular carcinoma (HCC), glioblastoma, and papillary thyroid carcinoma (PTC) (Ho et al., Oncotarget 7(31):49765-49776 (2016); Hsu et al., Oncotarget. 7(27):41748-41757 (2016); Liu et al., Biomed Pharmacother. 114:108605 (2019); and Varghese et al., Oncotarget 7:20140-51 (2016)). One publication reported that MAP4K3 immunohistochemistry (IHC) staining signals per square microns are decreased in cancer tissues of pancreatic cancer patients. Nevertheless, IHC staining intensity of MAP4K3 seems to be more condensed in ductal adenocarcinoma-like cells, which would be consistent with others' findings that MAP4K3 is overexpressed in several cancer tissues. Moreover, a MAP4K3 somatic mutation, encoding E351K, has been identified in human pancreatic cancer (Jones et al., Science 321:1801-6 (2008). The MAP4K3 E351K mutant displays higher kinase activity than that of wild-type MAP4K3, indicating that MAP4K3 is an oncogene involved in tumorigenesis of human pancreatic cancer. MAP4K3 may induce cancer metastasis/recurrence of NSCLC and HCC by enhancing cell migration/invasion (Chuang et al., Journal of Biomedical Science 26:82 (2019)). MAP4K3 overexpression may contribute to metastasis through proinflammatory cytokines. MAP4K3 overexpression in T cells induces production of IL-17A (Chuang et al., Sci. Adv. 4:eaat5401 (2018)), which promotes cancer cell migration and increases cancer metastasis. MAP4K3 overexpression is a therapeutic target for autoimmune diseases and cancer recurrence. Accordingly, inhibition of MAP4K3 may be useful for treating cancer patients.

Besides cell growth, cell proliferation, and cell migration, MAP4K3 also regulates animal lifespan. MAP4K3 deficiency in Caenorhabditis elegans results in an expansion of the worm lifespan. Similarly, MAP4K3-deficient mice show a significant extension of lifespan. The phenotypes of MAP4K3-deficient mice are normal and healthy. The serum levels of proinflammatory cytokines are increased in aged wild-type mice, but are decreased in aged MAP4K3-deficient mice. Chronic inflammation plays an important role in the aging processes. Thus, expanded lifespan of MAP4K3 deficient mice may be due to decreased inflammatory responses, suggesting that MAP4K3 inhibitor may have anti-aging effect.

Accordingly, inhibition of MAP4K3 could have immense therapeutic potential. Small molecule inhibitors of MAP4K3 could be developed into drugs for medicinal use in human patients suffering from neurological diseases or different types of cancer.

Although ATP-site small molecule kinase inhibitors to MAP4K3 have been discovered, such compounds were not further developed due to potential toxicities, stemming from off-target inhibition of other MAPK family members, which is a common problem when targeting the ATP binding pocket. When the MAP4K3 crystal structure was analyzed, it was noted that MAP4K3 exists as a dimer with ordered domain-swapped activation loops, with a-AL and a-EF helices. One monomer's activation loop is bound by a symmetry-mate such that the a-EF helix and loop of one monomer is in a pocket between the a-G helix and the activation loop of its symmetry-mate. It was postulated that a small molecule inhibitor occupying the dimer interface would disrupt the dimer or lock in the dimer, inhibiting enzyme activity.

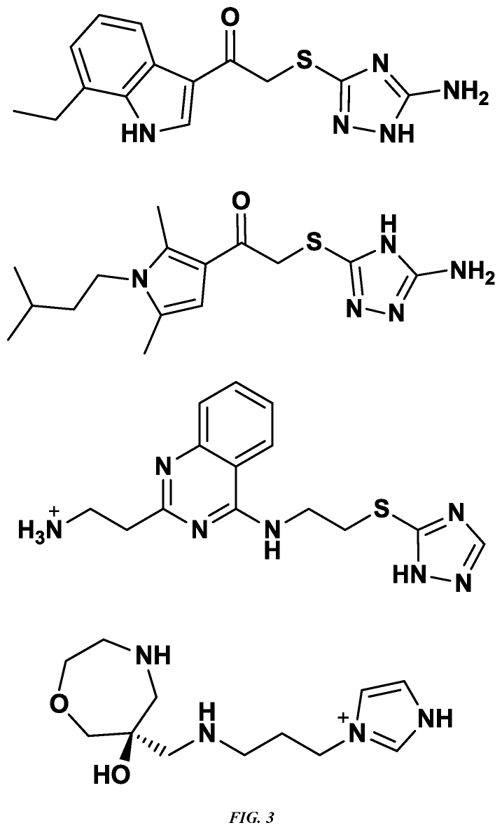

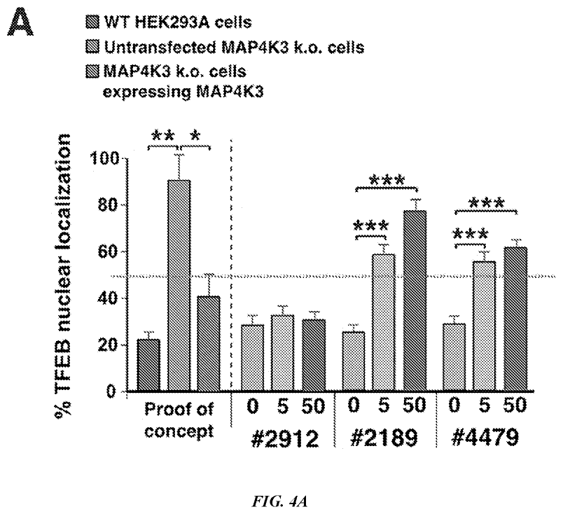

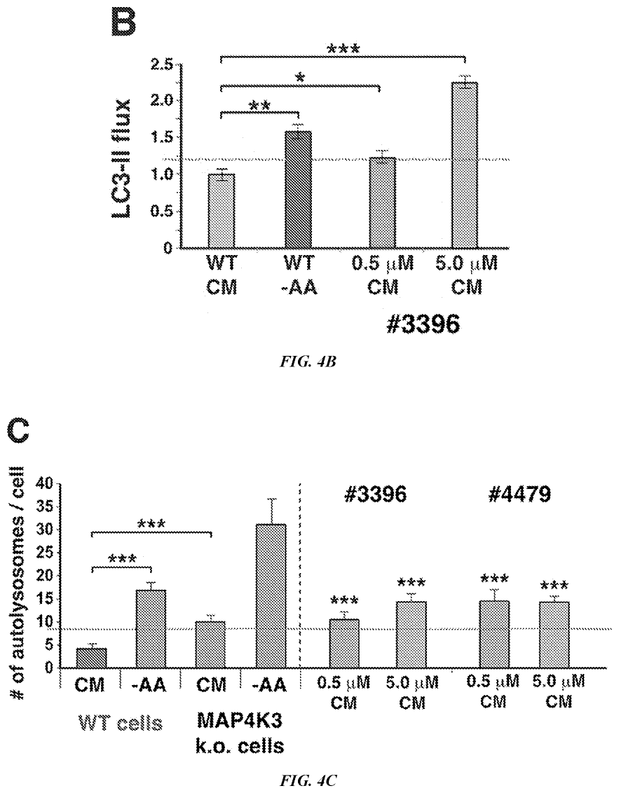

By leveraging in silico screening with medicinal chemistry a series of MAP4K3 inhibitor candidates were identified. In particular, in silico screening was carried out by using adjacent amino acids to create pharmacophores with SYBYL-X 2.0 software to screen a 2 million compound ZINC database [‘LEADS NOW’ 3] via Lipinski's rules, which yielded ˜2500 hits. Hits were interactively docked into the prospective binding pocket and scored. Compounds were prioritized based upon docking, and 13 compounds were selected for entry into a “important path” of secondary and tertiary screens (e.g., see FIG. 2). This analysis identified four “hits” capable of inhibiting MAP4K3 phosphorylation of PKC-theta by >40% (e.g., see FIG. 3). Furthermore, these four hits robustly promoted TFEB nuclear localization and potently induced autophagy flux (e.g., see FIG. 4). It was expected that additional, and possibly improved MAP4K3 inhibitors could be produced by incorporating common structural features seen in the four hit compounds.

The disclosure provides for compounds and compositions comprising an inhibitor of MAP4K3. In a particular embodiment, the disclosure provides a MAP4K3 inhibitor, or a composition comprising thereof, having the structure of Formula I:

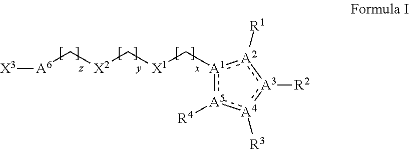

or a pharmaceutically acceptable salt, solvate, or prodrug thereof,
wherein,

A¹ is N⁺, C or CR⁵;

A² is N, C or CR⁶;

A³ is N, C or CR⁷;

A⁴ is N, C or CR⁸;

A⁵ is N, C or CR⁹;

A⁶ is a nitrogen containing optionally substituted heterocycle, wherein the nitrogen containing heterocycle can comprise one or more ring structures, including fused ring structures;

R¹-R⁴ are each individually absent or selected from H, D, optionally substituted (C₁-C₁₂)-alkyl, optionally substituted (C₁-C₁₂)-heteroalkyl, optionally substituted (C₂-C₁₂)-alkenyl, optionally substituted (C₂₋₁₂)-heteroalkenyl, optionally substituted (C₂₋₁₂)-alkynyl, optionally substituted (C₂-C₁₂)-heteroalkynyl, optionally substituted (C₃-C₁₂)-cycloalkyl, optionally substituted (C₄-C₁₂)-cycloalkenyl, halo (e.g., F, Cl, Br or I), optionally substituted oxygen containing functional group (e.g., hydroxyl, ketone, aldehyde, acyl halide, carbonate, carboxylic acid, ester, and ether), optionally substituted nitrogen containing functional group (e.g., amide, amine, imine, azide, cyanate, azo, nitrate, nitrile, nitro, and nitroso), optionally substituted sulfur containing functional group (e.g., thiol, sulfide, disulfide, sulfoxide, sulfone, sulfinic acid, sulfonic acid, thiocyanate, thione, and thial), optionally substituted phosphorous containing functional group (e.g., phosphine, phosphonic acid, phosphate, phosphodiester), optionally substituted boron containing functional group (e.g., boronic acid, boronic ester, borinic acid, and borinic ester), optionally substituted aryl, optionally substituted heterocycle;

R⁵-R⁹ are each individually selected from H, D, optionally substituted (C₁-C₁₂)-alkyl, optionally substituted (C₁-C₁₂)-heteroalkyl, optionally substituted (C₂-C₁₂)-alkenyl, optionally substituted (C₂₋₁₂)-heteroalkenyl, optionally substituted (C₂₋₁₂)-alkynyl, optionally substituted (C₂-C₁₂)-heteroalkynyl, optionally substituted (C₃-C₁₂)-cycloalkyl, optionally substituted (C₄-C₁₂)-cycloalkenyl, halo (e.g., F, Cl, Br or I), optionally substituted oxygen containing functional group (e.g., hydroxyl, ketone, aldehyde, acyl halide, carbonate, carboxylic acid, ester, and ether), optionally substituted nitrogen containing functional group (e.g., amide, amine, imine, azide, cyanate, azo, nitrate, nitrile, nitro, and nitroso), optionally substituted sulfur containing functional group (e.g., thiol, sulfide, disulfide, sulfoxide, sulfone, sulfinic acid, sulfonic acid, thiocyanate, thione, and thial), optionally substituted phosphorous containing functional group (e.g., phosphine, phosphonic acid, phosphate, phosphodiester), optionally substituted boron containing functional group (e.g., boronic acid, boronic ester, borinic acid, and borinic ester), optionally substituted aryl, optionally substituted heterocycle;

R¹⁰-R¹⁴ are each individually selected from H, D, halo, and an optionally substituted (C₁-C₆)-alkyl;

X¹ is S, O, CR¹⁰R¹¹ or NR¹²;

X² is NR¹³,

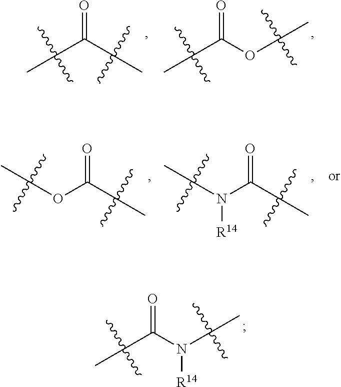

X³ is selected from H, D, halo, hydroxyl, amine, optionally substituted (C₁-C₆)-alkyl, azido, nitrile, and a (C₁-C₆)-alkylammonium group;

x is an integer selected from 0, 1, 2, or 3;

y is an integer selected from 0, 1, 2 or 3;

z is an integer selected from 0, 1, 2, or 3;

wherein, at least 2 of A¹-A⁵ comprise nitrogen containing groups,
wherein, there is only one double bond located between one of the following recited groups: A¹ and A⁵, and A¹ and A²; and wherein, there is only one double bond located between one of the following recited groups: A² and A³, A³ and A⁴, and A⁴ and A⁵.
   In yet a further embodiment, A⁶ is selected from

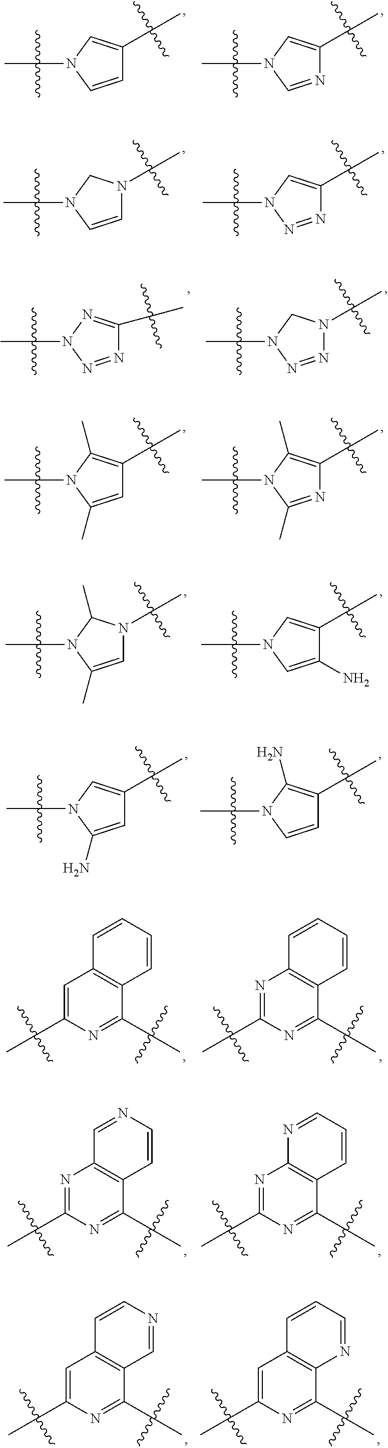

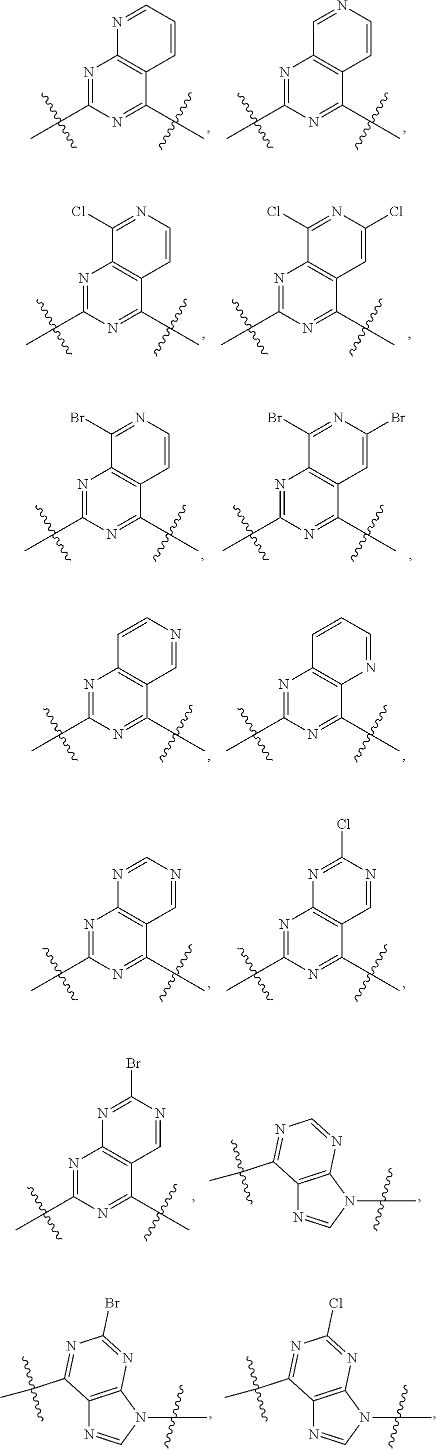

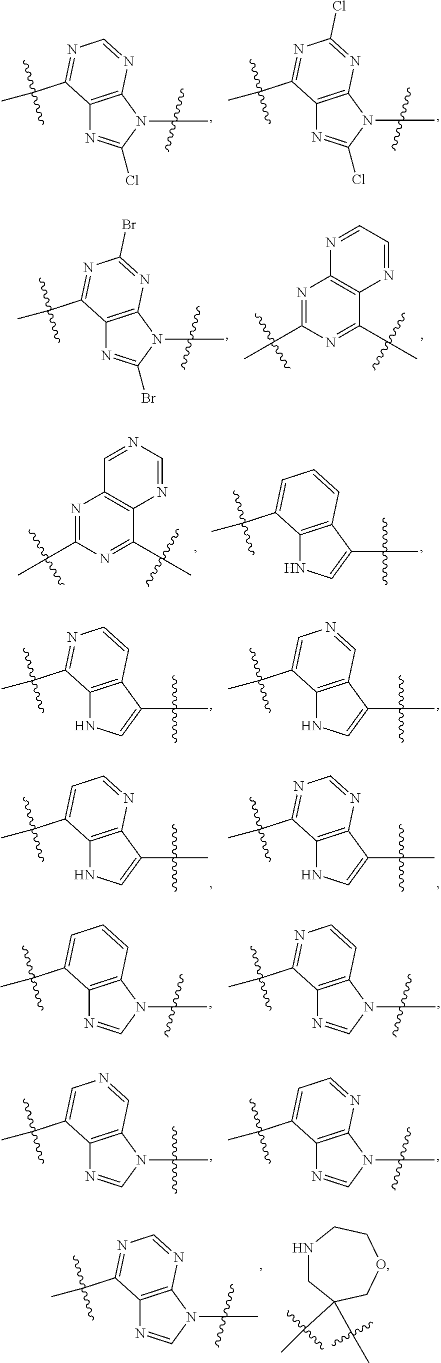

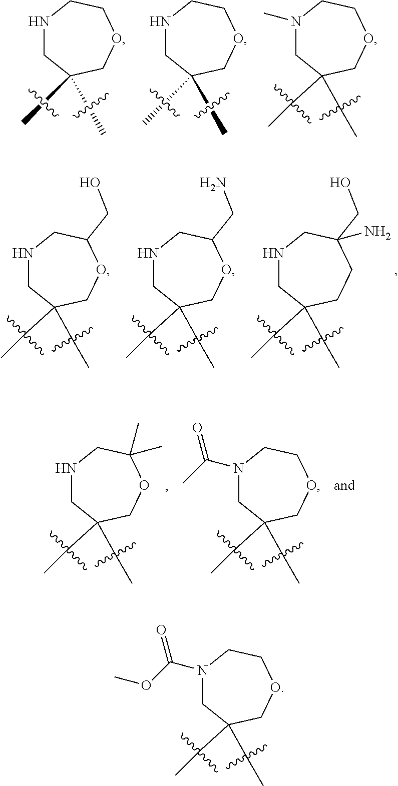

In a particular embodiment, the MAP4K3 inhibitor of Formula I does not have a structure selected from:

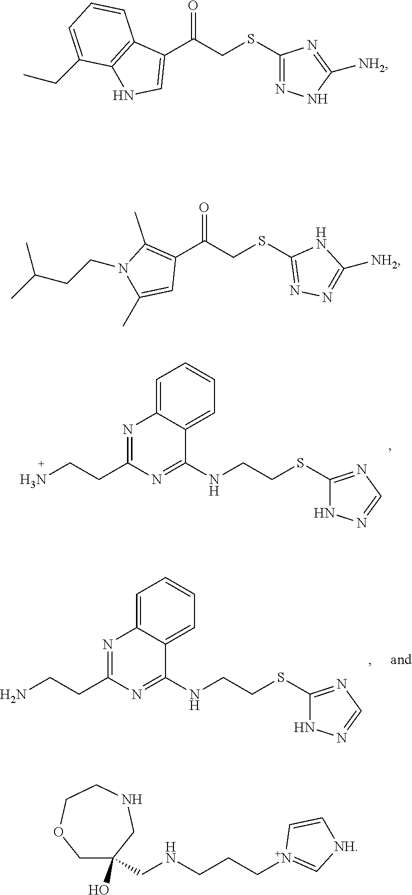 [SMILES: CCc1cccc2c(C(=O)CSc3n[nH]c(N)n3)c[nH]c12 ; Cc1cc(C(=O)CSc2nnc(N)[nH]2)c(C)n1CCC(C)C ; NCCc1nc(NCCSc2ncn[nH]2)c2ccccc2n1 ; NCCc1nc(NCCSc2ncn[nH]2)c2ccccc2n1 ; O[C@]1(CNCCC[n+]2cc[nH]c2)CNCCOC1]

In another embodiment, the MAP4K3 inhibitor comprising the structure of Formula I inhibits MAP4K3 phosphorylation of PKC-theta by at least 5%, 10%, 15%, 20%, 25%, 30%, 35%, 40%, 45%, 50%, 55%, 60%, 65%, 70%, 75%, 80%, 85%, or 90%, or a range that includes or is between any two of the foregoing percentages.

In a particular embodiment, the disclosure provides a MAP4K3 inhibitor, or a composition comprising thereof, having the structure of Formula I(a):

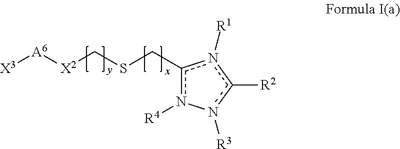

or a pharmaceutically acceptable salt, solvate, or prodrug thereof,
wherein,

A⁶ is a nitrogen containing optionally substituted heterocycle, wherein the nitrogen containing heterocycle can comprise one or more ring structures, including fused ring structures;

R¹-R⁴ are each individually absent or selected from H, D, optionally substituted (C₁-C₆)-alkyl, optionally substituted (C₁-C₆)-heteroalkyl, optionally substituted (C₂-C₆)-alkenyl, optionally substituted (C₂₋₆)-heteroalkenyl, optionally substituted (C₂₋₆)-alkynyl, optionally substituted (C₂-C₆)-heteroalkynyl, optionally substituted (C₃-C₈)-cycloalkyl, optionally substituted (C₄-C₈)-cycloalkenyl, halo (e.g., F, Cl, Br or I), optionally substituted oxygen containing functional group (e.g., hydroxyl, ketone, aldehyde, acyl halide, carbonate, carboxylic acid, ester, and ether), optionally substituted nitrogen containing functional group (e.g., amide, amine, imine, azide, cyanate, azo, nitrate, nitrile, nitro, and nitroso), optionally substituted sulfur containing functional group (e.g., thiol, sulfide, disulfide, sulfoxide, sulfone, sulfinic acid, sulfonic acid, thiocyanate, thione, and thial), optionally substituted phosphorous containing functional group (e.g., phosphine, phosphonic acid, phosphate, phosphodiester), optionally substituted boron containing functional group (e.g., boronic acid, boronic ester, borinic acid, and borinic ester), optionally substituted aryl, and optionally substituted heterocycle;

R¹³-R¹⁴ are each individually selected from H, D, halo, and an optionally substituted (C₁-C₆)-alkyl;

X² is NR¹³,

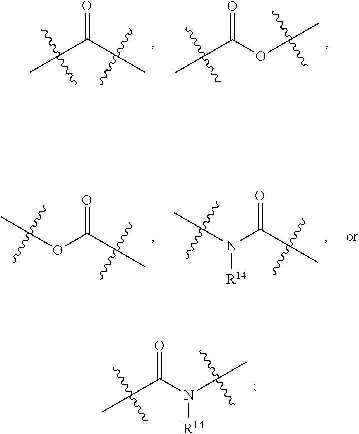

X³ is selected from H, D, halo, hydroxyl, amine, optionally substituted (C₁-C₆)-alkyl, azido, nitrile, and a (C₁-C₆)-alkylammonium group;

x is an integer selected from 0 or 1;

y is an integer selected from 1, 2, or 3; and

z is an integer selected from 0 or 1. In yet a further embodiment, A⁶ is selected from

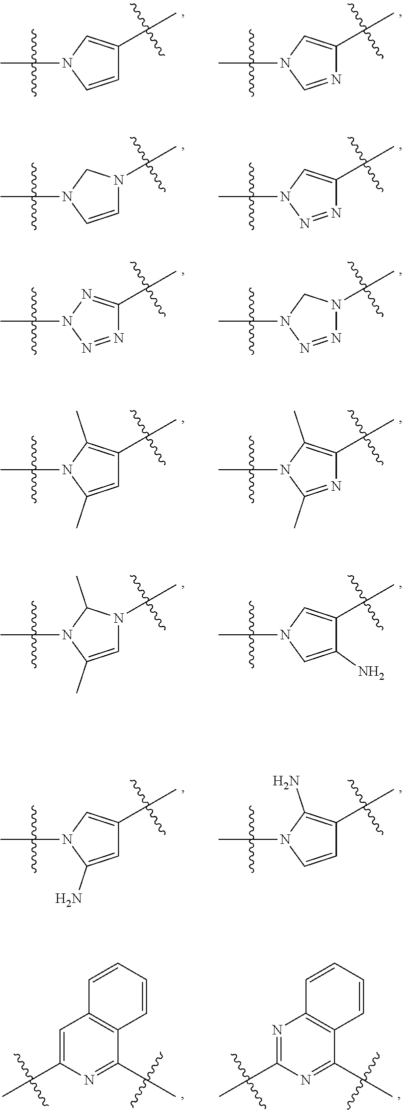

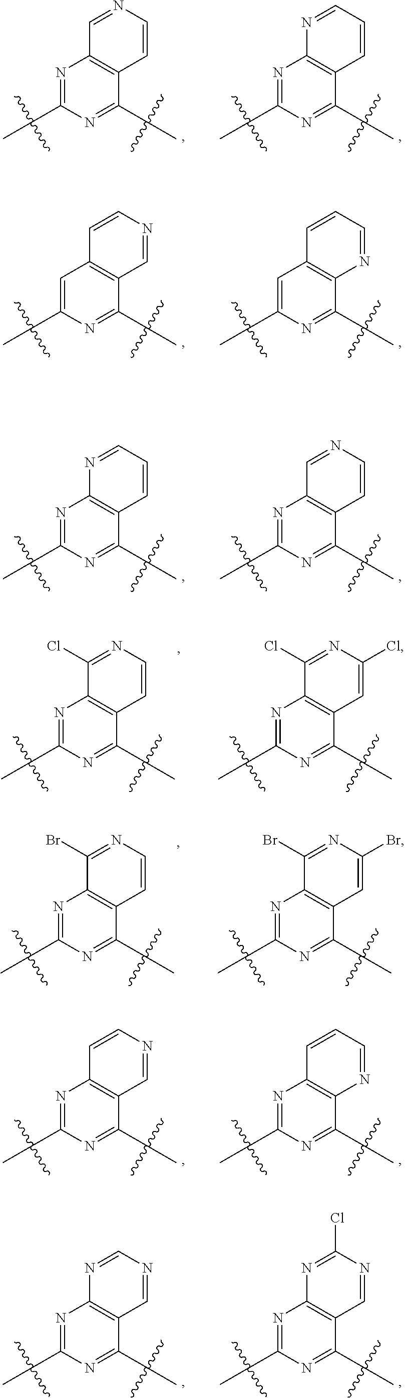

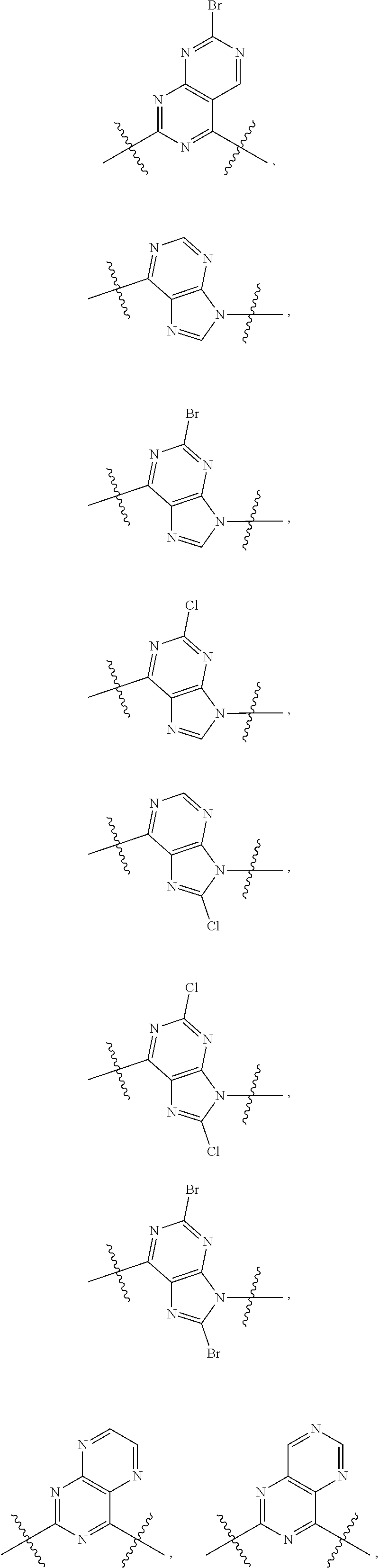

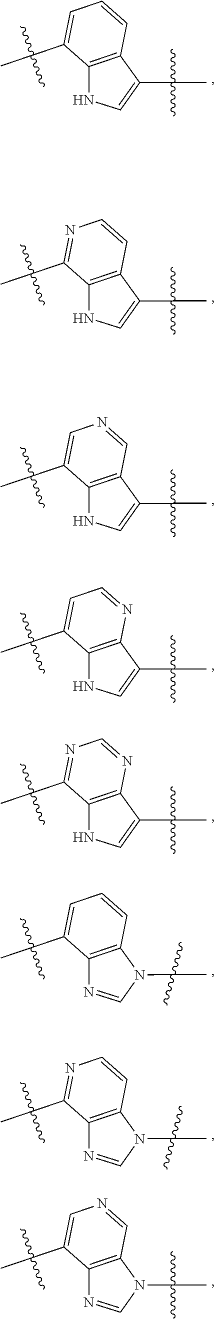

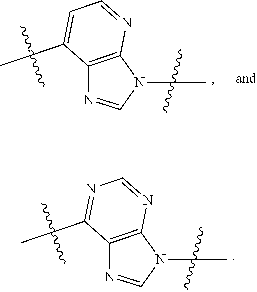 [SMILES: *c1ccnc2c1ncn2* ; *c1ncnc2c1ncn2*]

In a particular embodiment, the MAP4K3 inhibitor of Formula I(a) does not have a structure selected from:

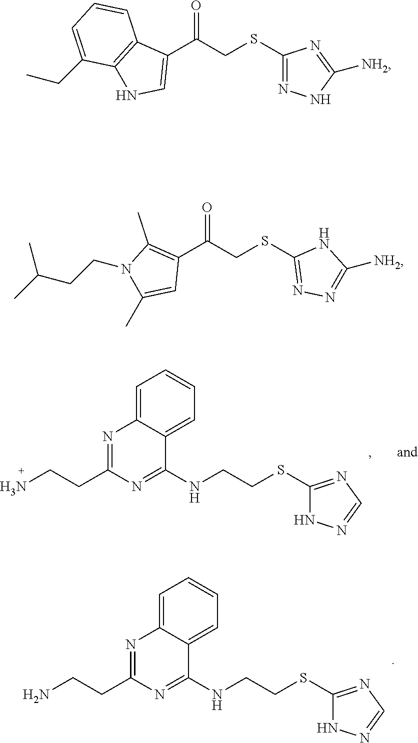 [SMILES: CCc1cccc2c(C(=O)CSc3n[nH]c(N)n3)c[nH]c12 ; Cc1cc(C(=O)CSc2nnc(N)[nH]2)c(C)n1CCC(C)C ; NCCc1nc(NCCSc2ncn[nH]2)c2ccccc2n1 ; NCCc1nc(NCCSc2ncn[nH]2)c2ccccc2n1]

In another embodiment, the MAP4K3 inhibitor comprising the structure of Formula I(a) inhibits MAP4K3 phosphorylation of PKCθThr538/total PKCθ by at least 5%, 10%, 15%, 20%, 25%, 30%, 35%, 40%, 45%, 50%, 55%, 60%, 65%, 70%, 75%, 80%, 85%, 90%, or a range that includes or is between any two of the foregoing percentages.

In a particular embodiment, the disclosure provides a MAP4K3 inhibitor, or a composition comprising thereof, having the structure of Formula I(b):

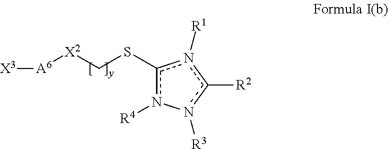

or a pharmaceutically acceptable salt, solvate, or prodrug thereof,
wherein,

A⁶ is selected from

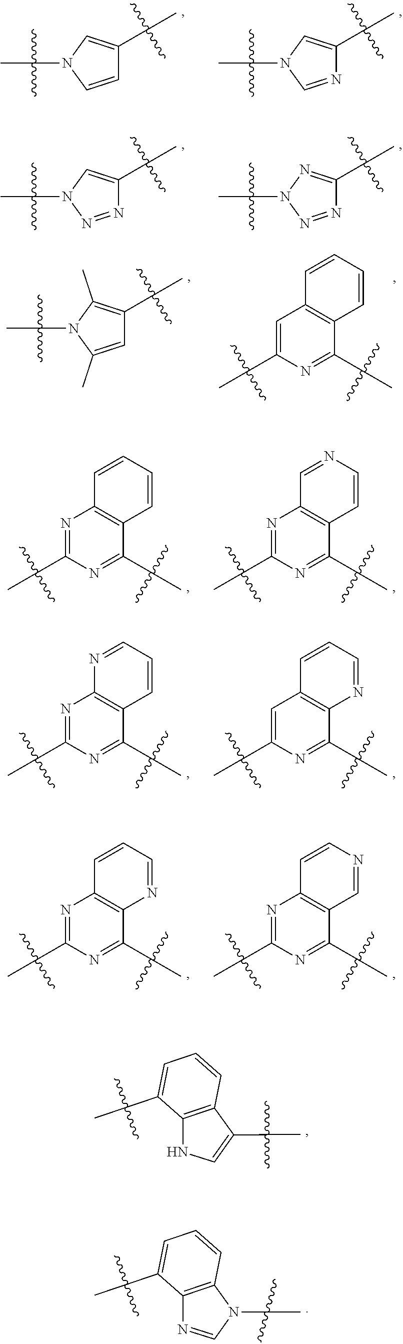

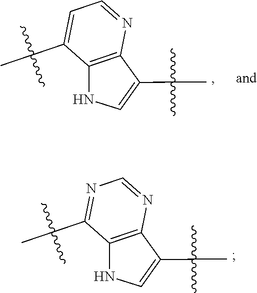 [SMILES: *c1c[nH]c2c(*)ccnc12 ; *c1c[nH]c2c(*)ncnc12]

R¹-R⁴ are each individually absent or selected from H, D, optionally substituted (C₁-C₆)-alkyl, optionally substituted (C₁C)-heteroalkyl, optionally substituted (C₂—C)-alkenyl, optionally substituted (C₂₋₆)-heteroalkenyl, optionally substituted (C₂₋₆)-alkynyl, optionally substituted (C₂-C₆)-heteroalkynyl, optionally substituted (C₃-C₈)-cycloalkyl, optionally substituted (C₄-C₈)-cycloalkenyl, halo (e.g., F, Cl, Br or I), optionally substituted oxygen containing functional group (e.g., hydroxyl, ketone, aldehyde, acyl halide, carbonate, carboxylic acid, ester, and ether), optionally substituted nitrogen containing functional group (e.g., amide, amine, imine, azide, cyanate, azo, nitrate, nitrile, nitro, and nitroso), optionally substituted sulfur containing functional group (e.g., thiol, sulfide, disulfide, sulfoxide, sulfone, sulfinic acid, sulfonic acid, thiocyanate, thione, and thial), optionally substituted phosphorous containing functional group (e.g., phosphine, phosphonic acid, phosphate, phosphodiester), optionally substituted boron containing functional group (e.g., boronic acid, boronic ester, borinic acid, and borinic ester), optionally substituted aryl, and optionally substituted heterocycle;

R¹³ is selected from H, D, halo, and an optionally substituted (C₁-C₆)-alkyl;

X² is NR¹³ or

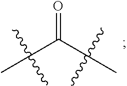 [SMILES: *C(*)=O]

X³ is an optionally substituted (C₁-C₆)-alkyl or a (C₁-C₆)-alkylammonium group; and

y is an integer selected from 1, 2, or 3. In a particular embodiment, the MAP4K3 inhibitor of Formula I(b) does not have a structure selected from:

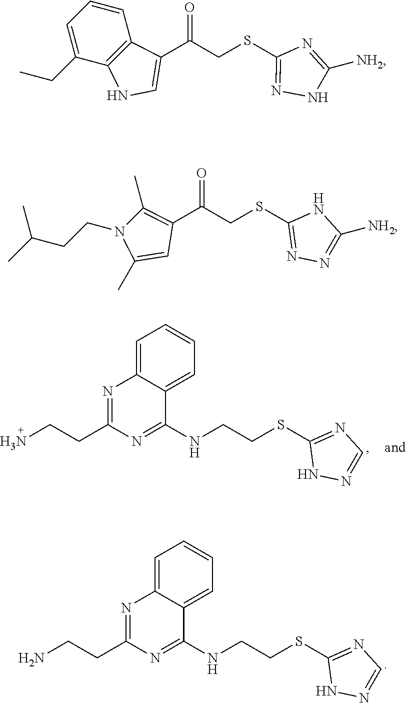 [SMILES: CCc1cccc2c(C(=O)CSc3n[nH]c(N)n3)c[nH]c12 ; Cc1cc(C(=O)CSc2nnc(N)[nH]2)c(C)n1CCC(C)C ; NCCc1nc(NCCSc2ncn[nH]2)c2ccccc2n1 ; NCCc1nc(NCCSc2ncn[nH]2)c2ccccc2n1]

In another embodiment, the MAP4K3 inhibitor comprising the structure of Formula I(b) inhibits MAP4K3 phosphorylation of PKCθThr538/total PKCθ by at least 5%, 10%, 15%, 20%, 25%, 30%, 35%, 40%, 45%, 50%, 55%, 60%, 65%, 70%, 75%, 80%, 85%, 90%, or a range that includes or is between any two of the foregoing percentages.

In a particular embodiment, the disclosure provides a MAP4K3 inhibitor, or a composition comprising thereof, having the structure of Formula I(c):

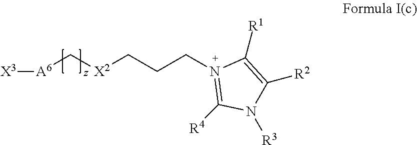

or a pharmaceutically acceptable salt, solvate, or prodrug thereof,
wherein,

A⁶ is selected from

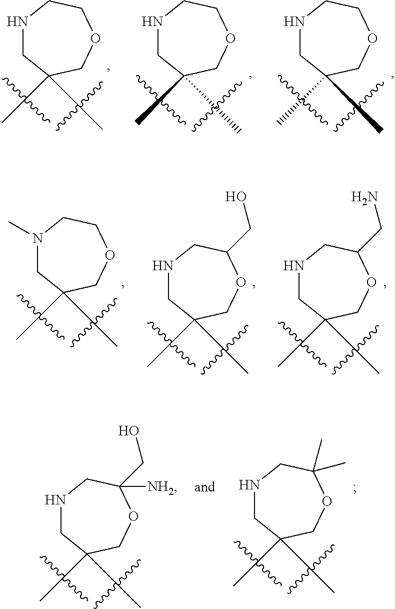

R¹³-R¹⁴ are each individually selected from H, D, halo, and an optionally substituted (C₁-C₆)-alkyl;

X² is selected from NR¹³,

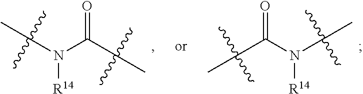

X³ is selected from H, D, halo, hydroxyl, amine, azido, and a nitrile;

z is an integer selected from 1, 2, or 3. In a further embodiment, the MAP4K3 inhibitor of Formula I(c) does not have a structure of:

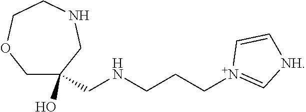 [SMILES: O[C@]1(CNCCC[n+]2cc[nH]c2)CNCCOC1]

In another embodiment, the MAP4K3 inhibitor comprising the structure of Formula I(c) inhibits MAP4K3 phosphorylation of PKCθThr538/total PKCθ by at least 5% o, 10%, 15% o, 20% o, 25%, 30%, 35%, 40%, 45%, 50%, 55%, 60%, 65%, 70%, 75%, 80%, 85%, 90%, or a range that includes or is between any two of the foregoing percentages.

In a further embodiment, the disclosure provides for a MAP4K3 inhibitor that has a structure selected from (a), (b), (c), (d) and (e):

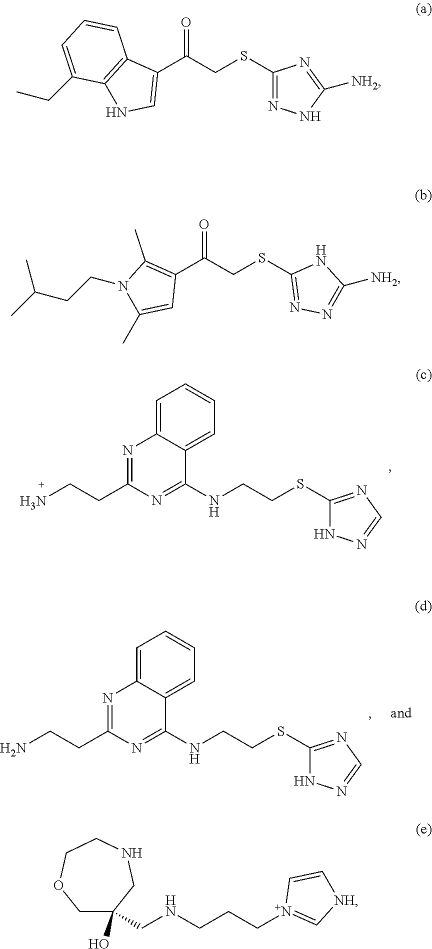 [SMILES: CCc1cccc2c(C(=O)CSc3n[nH]c(N)n3)c[nH]c12 ; Cc1cc(C(=O)CSc2nnc(N)[nH]2)c(C)n1CCC(C)C ; NCCc1nc(NCCSc2ncn[nH]2)c2ccccc2n1 ; NCCc1nc(NCCSc2ncn[nH]2)c2ccccc2n1 ; O[C@]1(CNCCC[n+]2cc[nH]c2)CNCCOC1]

or a pharmaceutically acceptable salt, solvate, or prodrug thereof. In another embodiment, the MAP4K3 inhibitor having a structure selected from (a), (b), (c) and (d) that inhibits MAP4K3 phosphorylation of PKCθThr538/total PKCθ by at least 5%, 10%, 15%, 20%, 25%, 30%, 35%, 40%, 45%, 50%, 55%, 60%, 65%, 70%, 75%, 80%, 85%, 90%, or a range that includes or is between any two of the foregoing percentages.

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(TS)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (+)-DL-lactic acid, lactobionic acid, lauric acid, maleic acid, (−)-L-malic acid, malonic acid, (+)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

The MAP4K3 inhibitors may also be provided as a prodrug, which is a functional derivative of the multi-targeting agent and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in “Transport Processes in Pharmaceutical Systems,” Amidon et al., Ed., Marcell Dekker, 185-218, 2000; Balant et al., Eur. J Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J Pharm. Sci. 1983, 72, 324-325; Freeman et al., J Chem. Soc., Chem. Commun. 1991, 875-877; Friis and Bundgaard, Eur. J Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; Waller et al., Br. J. Clin. Pharmac. 1989, 28, 497-507.

The disclosure further provides for a pharmaceutical composition which comprises a MAP4K3 inhibitor disclosed herein. Moreover, the pharmaceutical composition can be formulated into a form suitable for administration to a subject including the use of carriers, excipients, additives, or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, anti-oxidants, chelating agents, and inert gases. Other pharmaceutically acceptable carriers include aqueous solutions, non-toxic excipients, including salts, preservatives, buffers and the like, as described, for instance, in Remington's Pharmaceutical Sciences, 15th ed., Easton: Mack Publishing Co., 1405-1412, 1461-1487 (1975), and The National Formulary XIV., 14th ed., Washington: American Pharmaceutical Association (1975), the contents of which are hereby incorporated by reference. The pH and exact concentration of the various components of the pharmaceutical composition are adjusted according to routine skills in the art. See Goodman and Gilman's, The Pharmacological Basis for Therapeutics (7th ed.).

The pharmaceutical compositions according to the disclosure may be administered at a therapeutically effective amount either locally or systemically. As used herein, “administering a therapeutically effective amount” is intended to include methods of giving or applying a pharmaceutical composition of the disclosure to a subject that allow the composition to perform its intended therapeutic function. The therapeutically effective amounts will vary according to factors, such as the degree of infection in a subject, the age, sex, and weight of the individual. Dosage regime can be adjusted to provide the optimum therapeutic response. For example, several divided doses can be administered daily or the dose can be proportionally reduced as indicated by the exigencies of the therapeutic situation.

The pharmaceutical composition can be administered in a convenient manner, such as by injection (e.g., subcutaneous, intravenous, and the like), oral administration, inhalation, transdermal application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. The composition will typically be sterile and fluid to the extent that easy syringability exists. Typically, the composition will be stable under the conditions of manufacture and storage and preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, using a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols, such as mannitol, sorbitol, or sodium chloride are used in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

The pharmaceutical composition can be orally administered, for example, with an inert diluent or an assimilable edible carrier. The pharmaceutical composition and other ingredients can also be enclosed in a hard or soft-shell gelatin capsule, compressed into tablets, or incorporated directly into the subject's diet. For oral therapeutic administration, the pharmaceutical composition can be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations can, of course, be varied and can conveniently be between about 5% to about 80% of the weight of the unit.

The tablets, troches, pills, capsules, and the like can also contain the following: a binder, such as gum tragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Of course, any material used in preparing any dosage unit form should be pharmaceutically pure and substantially non-toxic in the amounts employed. In addition, the pharmaceutical composition can be incorporated into sustained-release preparations and formulations. Thus, a “pharmaceutically acceptable carrier” is intended to include solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the pharmaceutical composition, use thereof in the therapeutic compositions and methods of treatment is contemplated. Supplementary active compounds can also be incorporated into the compositions.

It is especially advantageous to formulate parenteral compositions in dosage unit form for ease of administration and uniformity of dosage. “Dosage unit form” as used herein, refers to physically discrete units suited as unitary dosages for the subject to be treated; each unit containing a predetermined quantity of pharmaceutical composition is calculated to produce the desired therapeutic effect in association with the required pharmaceutical carrier. The specification for the dosage unit forms of the disclosure is related to the characteristics of the pharmaceutical composition and the desired therapeutic effect.

The principal pharmaceutical composition is compounded for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

The disclosure further provides methods for treating a targeted disorder in a subject, comprising: administering a therapeutically effective amount of a MAP4K3 inhibitor disclosed herein to the subject in need thereof. As used herein, the term “therapeutically effective amount” refers to an amount which, when administered in a proper dosing regimen, is sufficient to treat (therapeutically or prophylactically) the target disorder. For example, and effective amount is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

In one embodiment, an effective amount of a MAP4K3 inhibitor of the disclosure can range from about 1 mg to about 6000 mg per treatment. In more specific embodiments the range is from about 10 to 3000 mg, or from 20 to 1200 mg, or most specifically from about 100 to 600 mg per treatment. Treatment typically is administered twice daily. Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician.

Generally, the disorder targeted to be treated with a MAP4K3 inhibitor of the disclosure is a disorder associated with overexpression of MAP4K3, mis-regulation of MAP4K3, abnormal levels of MAP4K3, and/or abnormal activity of MAP4K3 and its target mTORC1. Such disorders can include disorders associated with abnormal cell growth, cell proliferation, cell migration, and aging. More specifically, the disorders include, but are not limited to, cancer, neurological diseases, autoimmune disorders, and aging. Examples of cancers treatable by a MAP4K3 inhibitor disclosed herein include, but are not limited to, renal cell carcinoma (RCC), human non-small cell lung carcinoma (NSCLC), hepatocellular carcinoma (HCC), glioblastoma, and papillary thyroid carcinoma (PTC). Examples of autoimmune disorders treatable by a MAP4K3 inhibitor disclosed herein include, but are not limited to, collagen induced arthritis (CIA), experimental autoimmune encephalomyelitis (EAE), inflammatory bowel disease (IBD), systemic lupus erythematosus (SLE), rheumatoid arthritis (RA), multiple sclerosis (MS), Type 1 diabetes mellitus, Guillain-Barre syndrome, psoriasis, chronic inflammatory demyelinating polyneuropathy, Graves' disease, Hashimoto's thyroiditis, myasthenia gravis, vasculitis, Sjögren syndrome, Addison disease, celiac disease, dermatomyositis, and adult-onset Still's disease (AOSD). Examples of neurological diseases treatable by a MAP4K3 inhibitor disclosed herein include, but are not limited to, tuberous sclerosis, epilepsy, Fragile X syndrome, Down syndrome, Rett syndrome, Alzheimer disease, Parkinson disease, and Huntington disease.

The MAP4K3 inhibitors disclosed herein may also be combined or used in combination with other agents useful in the treatment of MAP4K3-mediated disorders. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced).

Such other agents, adjuvants, or drugs, may be administered, by a route and in an amount commonly used therefor, simultaneously or sequentially with a compound as disclosed herein. When a compound as disclosed herein is used contemporaneously with one or more other drugs, a pharmaceutical composition containing such other drugs in addition to the compound disclosed herein may be utilized, but is not required.

In a certain embodiment, a MAP4K3 inhibitor disclosed herein is used in combination with one or more immunosuppressive agents. Examples of immunosuppressive agents include, but are not limited to, glucocorticoids, such as prednisone, dexamethasone, and hydrocortisone; cytostatics, such as purine analogs; alkylating agents, such as cyclophosphamide, nitrosoureas, platinum compounds; antimetabolites, such as methotrexate, azathioprine, mercaptopurine, fluorouracil; anti-CD20 antibodies; anti-CD25 antibodies; anti-CD3 antibodies; muromonab-CD3 antibody; ciclosporin; tacrolimus; sirolimus; everolimus; zotarolimus; interferons; TNFα binding proteins, such as infliximab, etanercept, and adalimumab; mycophenolic acid; and fingolimod.

In a particular embodiment, a MAP4K3 inhibitor disclosed herein is used in combination with one or more anticancer agents. Examples of anticancer agents, include, but are not limited to, rapamycin inhibitors, such as AFINITOR® (everolimus), ASTAGRAF®, ENCARSUS®, HECORIA®, and PROGRAF® tacrolimus; DNA damage response inhibitors, including especially PARP inhibitors, such as LYNPARZA® (Olaparib); immune checkpoint inhibitors, including anti-PD1, anti-PD-L1, and anti-CTLA-4 inhibitors, such as KEYTRUDA® pembrolizumab; immune checkpoint activators, including anti-CD40 and anti-OX-40 inhibitors; Chimeric Antigen Receptor (CAR) T-cell therapies; alkylating agents such as thiotepa and CYTOXAN® cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and tiimethylolomelamine; acetogenins (e.g., bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBT-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; vinca alkaloids; epipodophyllotoxins; antibiotics such as the enediyne antibiotics (e.g., calicheamicin, especially calicheamicin gammall and calicheamicin omegall; L-asparaginase; anthracenedione substituted urea; methyl hydrazine derivatives; dynemicin, including dynemicin A; bisphosphonates, such as clodronate; an esperamicin; as well as neocarzinostatin chromophore and related chromoprotein enediyne antibiotic chromophores), aclacinomysins, actinomycin, anthramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiopurine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolaevulinic acid; eniluracil; amacrine; bestrabucil; bisantrene; edatrexate; defofamine; demecolcine; diaziquone; eflornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantione; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2 2″-trichlorotiiethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; toxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® (docetaxel) (Rhone-Poulenc Rorer, Antony, France); chlorambucil; GEMZAR® (gemcitabine); 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DFMO); retinoids such as retinoic acid; capecitabine; leucovorin (LV); irinotecan; adrenocortical suppressant; adrenocorticosteroids; progestins; estrogens; androgens; gonadotropin-releasing hormone analogs; and pharmaceutically acceptable salts, acids or derivatives of any of the above. “Anticancer agents” also include anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON-toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASL® exemestane, formestanie, fadrozole, RIVISOR® vorozole, FEMARA® letrozole, and ARTMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in aberrant cell proliferation, such as, for example, PKC-alpha, Ralf and H-Ras; ribozymes such as a VEGF-A expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rJL-2; LURTOTECAN® topoisomerase 1 inhibitor; ABARELLX® rmRH; antibodies such as trastuzumab and pharmaceutically acceptable salts, acids or derivatives of any of the above.

The MAP4K3 inhibitors disclosed herein can also be administered in combination with other classes of compounds, including, but not limited to, norepinephrine reuptake inhibitors (NRIs) such as atomoxetine; dopamine reuptake inhibitors (DARIs), such as methylphenidate; serotonin-norepinephrine reuptake inhibitors (SNRIs), such as milnacipran; sedatives, such as diazepam; norepinephrine-dopamine reuptake inhibitor (NDRIs), such as bupropion; serotonin-norepinephrine-dopamine-reuptake-inhibitors (SNDRIs), such as venlafaxine; monoamine oxidase inhibitors, such as selegiline; hypothalamic phospholipids; endothelin converting enzyme (ECE) inhibitors, such as phosphoramidon; opioids, such as tramadol; thromboxane receptor antagonists, such as ifetroban; potassium channel openers; thrombin inhibitors, such as hirudin; hypothalamic phospholipids; growth factor inhibitors, such as modulators of PDGF activity; platelet activating factor (PAF) antagonists; anti-platelet agents, such as GPIIb/IIIa blockers (e.g., abdximab, eptifibatide, and tirofiban), P2Y(AC) antagonists (e.g., clopidogrel, ticlopidine and CS-747), and aspirin; anticoagulants, such as warfarin; low molecular weight heparins, such as enoxaparin; Factor VIIa Inhibitors and Factor Xa Inhibitors; renin inhibitors; neutral endopeptidase (NEP) inhibitors; vasopepsidase inhibitors (dual NEP-ACE inhibitors), such as omapatrilat and gemopatrilat; HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, or atorvastatin or visastatin); squalene synthetase inhibitors; fibrates; bile acid sequestrants, such as questran; niacin; anti-atherosclerotic agents, such as ACAT inhibitors; MTP Inhibitors; calcium channel blockers, such as amlodipine besylate; potassium channel activators; alpha-muscarinic agents; beta-muscarinic agents, such as carvedilol and metoprolol; antiarrhythmic agents; diuretics, such as chlorothiazide, hydrochlorothiazide, flumethiazide, hydroflumethiazide, bendroflumethiazide, methyl chlorothiazide, trichloromethiazide, polythiazide, benzothiazine, ethacrynic acid, ticrynafen, chlorthalidone, furosemide, mesalamine, bumetanide, triamterene, amiloride, and spironolactone; thrombolytic agents, such as tissue plasminogen activator (tPA), recombinant tPA, streptokinase, urokinase, prourokinase, and anisoylated plasminogen streptokinase activator complex (APSAC); anti-diabetic agents, such as biguanides (e.g. metformin), glucosidase inhibitors (e.g., acarbose), insulins, meglitinides (e.g., repaglinide), sulfonylureas (e.g., glimepiride, glyburide, and glipizide), thiazolidinediones (e.g. troglitazone, rosiglitazone and pioglitazone), and PPAR-gamma agonists; mineralocorticoid receptor antagonists, such as spironolactone and eplerenone; growth hormone secretagogues; aP2 inhibitors; phosphodiesterase inhibitors, such as PDE III inhibitors (e.g., cilostazol) and PDE V inhibitors (e.g., sildenafil, tadalafil, vardenafil); protein tyrosine kinase inhibitors; anti-inflammatories; antiproliferatives, such as methotrexate, FK506 (tacrolimus, Prograf), mycophenolate mofetil; chemotherapeutic agents; anticancer agents and cytotoxic agents (e.g., alkylating agents, such as nitrogen mustards, alkyl sulfonates, nitrosoureas, ethylenimines, and triazenes); antimetabolites, such as folate antagonists, purine analogues, and pyrridine analogues; antibiotics, such as anthracycline, bleomycin, mitomycin, dactinomycin, and plicamycin; enzymes, such as L-asparaginase; famesyl-protein transferase inhibitors; hormonal agents, such as estrogens/antiestrogens, androgens/antiandrogens, progestins, and luteinizing hormone-releasing hormone antagonists, and octreotide acetate; microtubule-disruptor agents, such as ecteinascidins; microtubule-stabilizing agents, such as paclitaxel, docetaxel, and epothilones A-F; plant-derived products, such as vinca alkaloids, epipodophyllotoxins, and taxanes; topoisomerase inhibitors; prenyl-protein transferase inhibitors; cyclosporins; steroids, such as prednisone and dexamethasone; cytotoxic drugs, such as azathioprine and cyclophosphamide; TNF-alpha inhibitors, such as tenidap; anti-TNF antibodies or soluble TNF receptor, such as etanercept, rapamycin, and leflunimide; cyclooxygenase-2 (COX-2) inhibitors, such as celecoxib and rofecoxib; and miscellaneous agents such as, hydroxyurea, procarbazine, mitotane, hexamethylmelamine, gold compounds, platinum coordination complexes, such as cisplatin, satraplatin, and carboplatin.

It is expected that some of the additional therapeutics and agents referenced above will act synergistically with a MAP4K3 inhibitor of the disclosure. When this occurs, it will allow the effective dosage of the additional therapeutic agent and/or the MAP4K3 inhibitor of the disclosure to be reduced from that required in a monotherapy. This has the advantage of minimizing toxic side effects of either the additional therapeutic agent or the MAP4K3 inhibitor of the disclosure, synergistic improvements in efficacy, improved ease of administration or use and/or reduced overall expense of compound preparation or formulation.

For use in the therapeutic or biological applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more MAP4K3 inhibitors described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself, a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

The disclosure further provides that the methods and compositions described herein can be further defined by the following aspects (aspects 1 to 18):

1. A MAP4K3 inhibitor comprising the structure of Formula I:

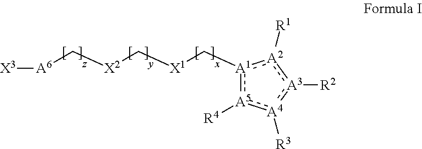

or a pharmaceutically acceptable salt, solvate, or prodrug thereof,
wherein,

A¹ is N⁺, C or CR⁵;

A² is N, C or CR⁶;

A³ is N, C or CR⁷;

A⁴ is N, C or CR⁸;

A⁵ is N, C or CR⁹;

A⁶ is a nitrogen containing optionally substituted heterocycle, wherein the nitrogen containing heterocycle can comprise one or more ring structures, including fused ring structures;

R¹-R⁴ are each individually absent or selected from H, D, optionally substituted (C₁. C₁₂)-alkyl, optionally substituted (C₁-C₁₂)-heteroalkyl, optionally substituted (C₂-C₁₂)-alkenyl, optionally substituted (C₂₋₁₂)-heteroalkenyl, optionally substituted (C₂₋₁₂)-alkynyl, optionally substituted (C₂-C₁₂)-heteroalkynyl, optionally substituted (C₃-C₁₂)-cycloalkyl, optionally substituted (C₄-C₁₂)-cycloalkenyl, optionally substituted aryl, optionally substituted heterocycle, halo, hydroxyl, ketone, aldehyde, acyl halide, carbonate, carboxylic acid, ester, ether, amide, amine, imine, azide, cyanate, azo, nitrate, nitrile, nitro, nitroso, thiol, sulfide, disulfide, sulfoxide, sulfone, sulfinic acid, sulfonic acid, thiocyanate, thione, thial, phosphine, phosphonic acid, phosphate, phosphodiester, boronic acid, boronic ester, borinic acid, and borinic ester;

R⁵-R⁹ are each individually selected from H, D, optionally substituted (C₁-C₁₂)-alkyl, optionally substituted (C₁-C₁₂)-heteroalkyl, optionally substituted (C₂-C₁₂)-alkenyl, optionally substituted (C₂₋₁₂)-heteroalkenyl, optionally substituted (C₂₋₁₂)-alkynyl, optionally substituted (C₂-C₁₂)-heteroalkynyl, optionally substituted (C₃-C₁₂)-cycloalkyl, optionally substituted (C₄-C₁₂)-cycloalkenyl, optionally substituted aryl, optionally substituted heterocycle, halo, hydroxyl, ketone, aldehyde, acyl halide, carbonate, carboxylic acid, ester, ether, amide, amine, imine, azide, cyanate, azo, nitrate, nitrile, nitro, nitroso, thiol, sulfide, disulfide, sulfoxide, sulfone, sulfinic acid, sulfonic acid, thiocyanate, thione, thial, phosphine, phosphonic acid, phosphate, phosphodiester, boronic acid, boronic ester, borinic acid, and borinic ester;

R¹⁰-R¹⁴ are each individually selected from H, D, halo, and an optionally substituted (C₁-C₆)-alkyl;

X¹ is S, O, CR¹⁰R¹¹ or NR¹²;

X² is NR¹³,

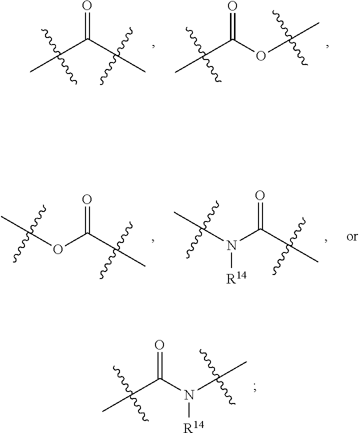

X³ is selected from H, D, halo, hydroxyl, amine, optionally substituted (C₁-C₆)-alkyl, azido, nitrile, and a (C₁-C₆)-alkylammonium group;

x is an integer selected from 0, 1, 2, or 3;

y is an integer selected from 0, 1, 2 or 3;

z is an integer selected from 0, 1, 2, or 3;

wherein, at least 2 of A¹-A⁵ comprise nitrogen containing groups,
wherein, there is only one double bond located between one of the following recited groups: A¹ and A⁵, and A¹ and A²; and wherein, there is only one double bond located between one of the following recited groups: A² and A³, A³ and A⁴, and A⁴ and A⁵,

particularly, wherein the nitrogen containing optionally substituted heterocycle of A⁶ is either unsubstituted or substituted with one or more of the following groups: (C₁-C₆)alkyl, (C₁-C₆)-heteroalkyl, (C₂-C₆)-alkenyl, (C₂₋₆)-heteroalkenyl, aryl, heterocycle, halo, hydroxyl, aldehyde, carboxylic acid, ester, ether, amide, amine, nitro, and thiol, more particularly, wherein the nitrogen containing optionally substituted heterocycle of A⁶ is either unsubstituted or substituted with one or more of the following groups: (C₁-C₆)alkyl, halo, hydroxyl, carboxylic acid, amine, nitro, and thiol; and/or

particularly, R¹-R⁴ are each individually absent or selected from H, D, optionally substituted (C₁-C₁₂)-alkyl, optionally substituted (C₁-C₁₂)-heteroalkyl, optionally substituted (C₂-C₁₂)-alkenyl, optionally substituted (C₃-C₈)-cycloalkyl, optionally substituted aryl, optionally substituted heterocycle, halo, hydroxyl, carboxylic acid, ester, ether, amide, amine, azide, nitro, and thiol; and/or

particularly, R⁵-R⁹ are each individually absent or selected from H, D, optionally substituted (C₁-C₁₂)-alkyl, optionally substituted (C₁-C₁₂)-heteroalkyl, optionally substituted (C₂-C₁₂)-alkenyl, optionally substituted (C₃-C₈)-cycloalkyl, optionally substituted aryl, optionally substituted heterocycle, halo, hydroxyl, carboxylic acid, ester, ether, amide, amine, azide, nitro, and thiol; and/or

particularly, X¹ is S, or CR¹⁰R¹¹; and/or

particularly, X² is NR¹³ or

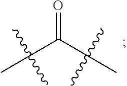 [SMILES: *C(*)=O]

and/or

particularly, X³ is selected from H, halo, hydroxyl, amine, optionally substituted (C₁-C₆)-alkyl, and a (C₁-C₆)-alkylammonium group; and/or

particularly, wherein there are double bonds between A^{t} and A⁵ and between A³ and A⁴, or between A^{t} and A⁵ and between A² and A³, or between A^{t} and A² and between A³ and A⁴, and/or

particularly, wherein the MAP4K3 inhibitor does not have a structure selected from:

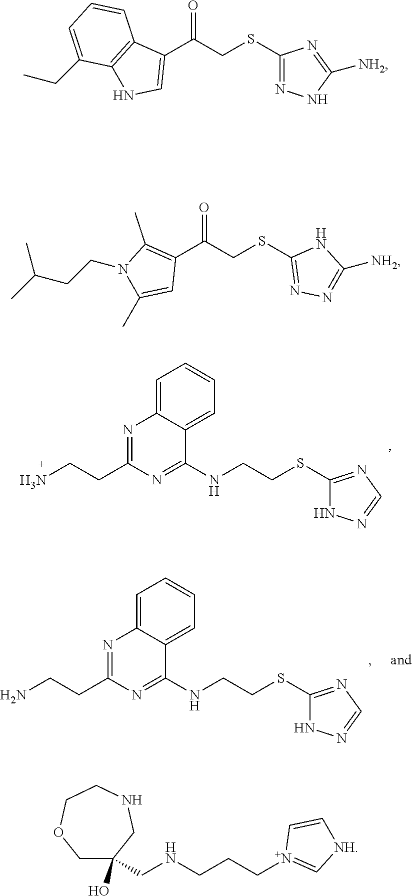 [SMILES: CCc1cccc2c(C(=O)CSc3n[nH]c(N)n3)c[nH]c12 ; Cc1cc(C(=O)CSc2nnc(N)[nH]2)c(C)n1CCC(C)C ; NCCc1nc(NCCSc2ncn[nH]2)c2ccccc2n1 ; NCCc1nc(NCCSc2ncn[nH]2)c2ccccc2n1 ; O[C@]1(CNCCC[n+]2cc[nH]c2)CNCCOC1]

2. The MAPK3 inhibitor of aspect 1, wherein A⁶ is selected from:

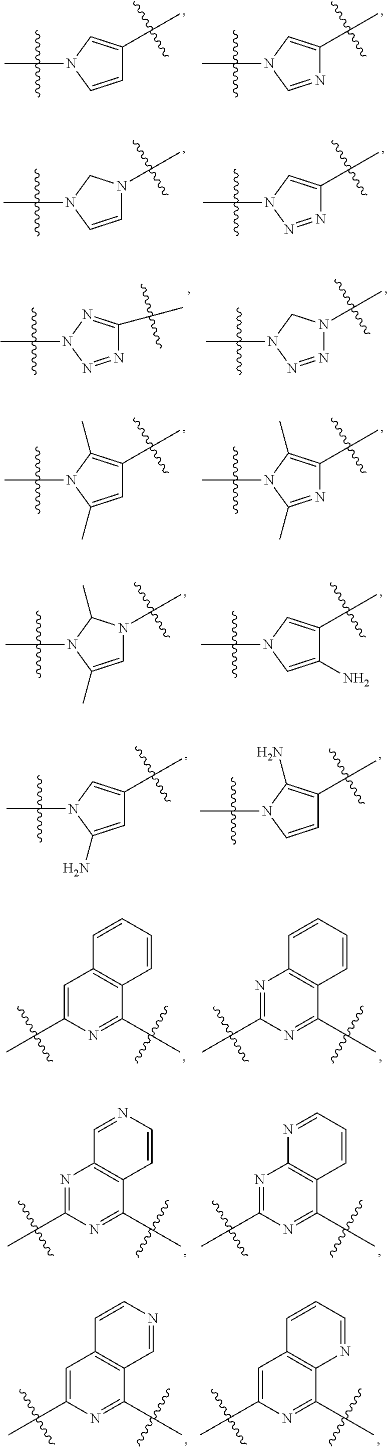

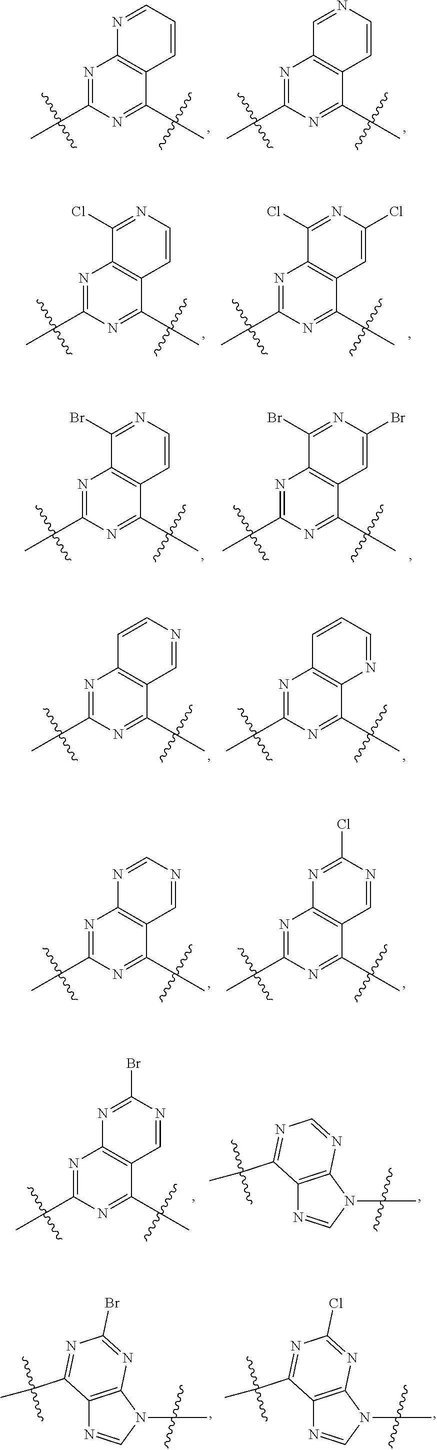

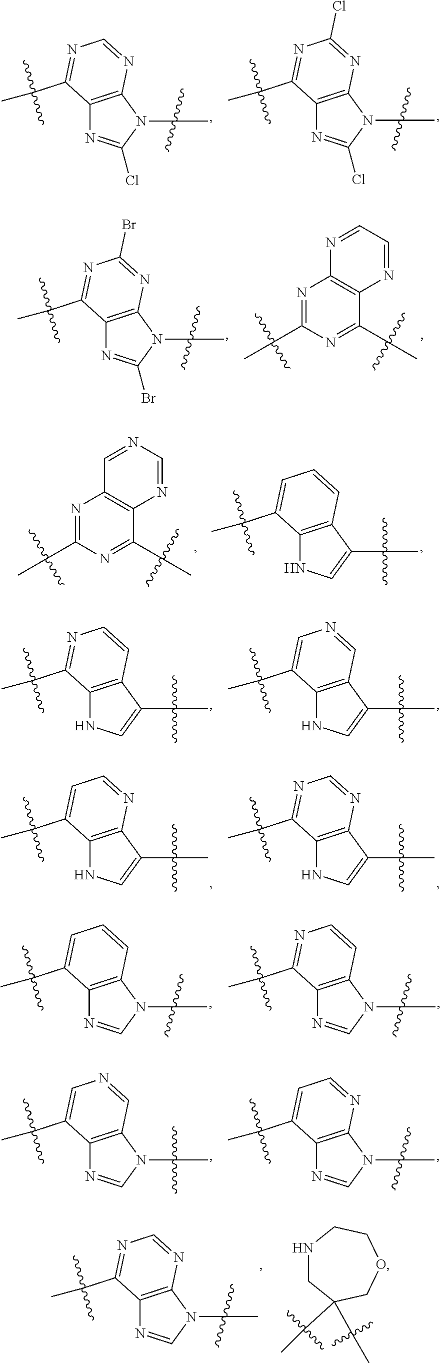

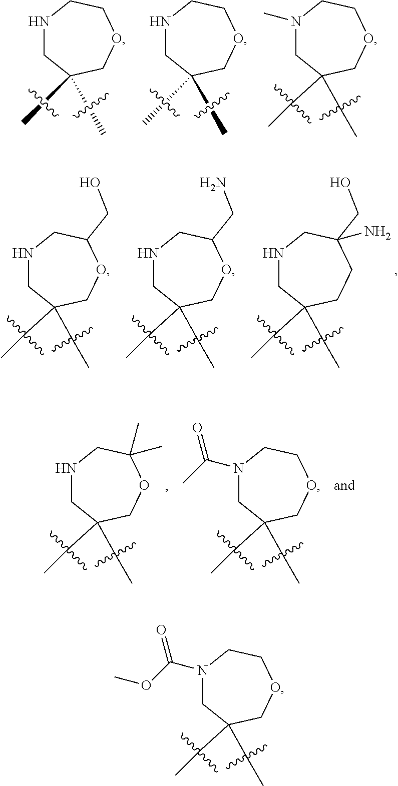

particularly, A⁶ is selected from:

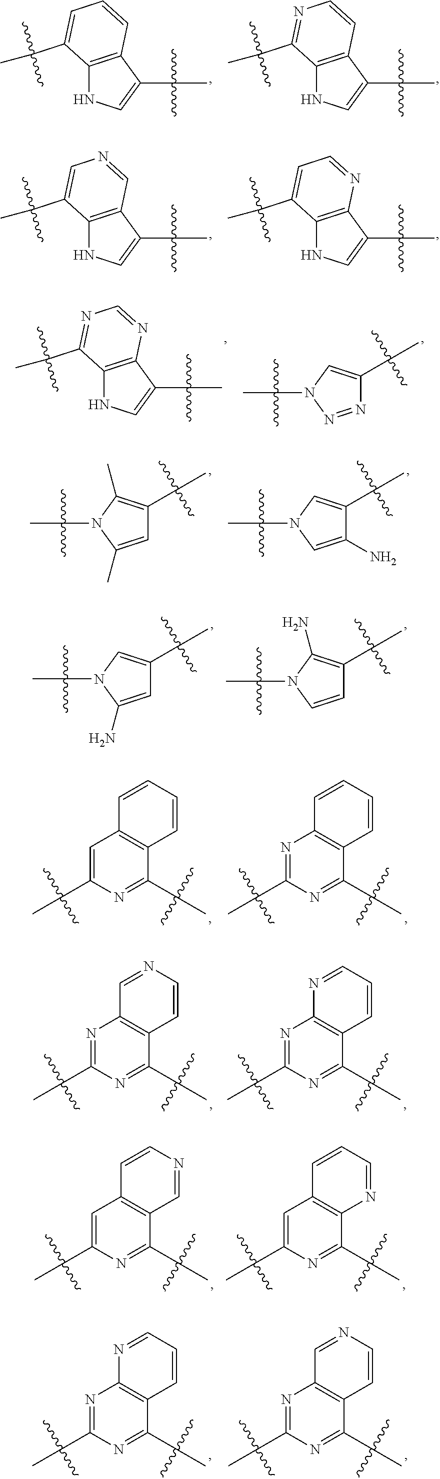

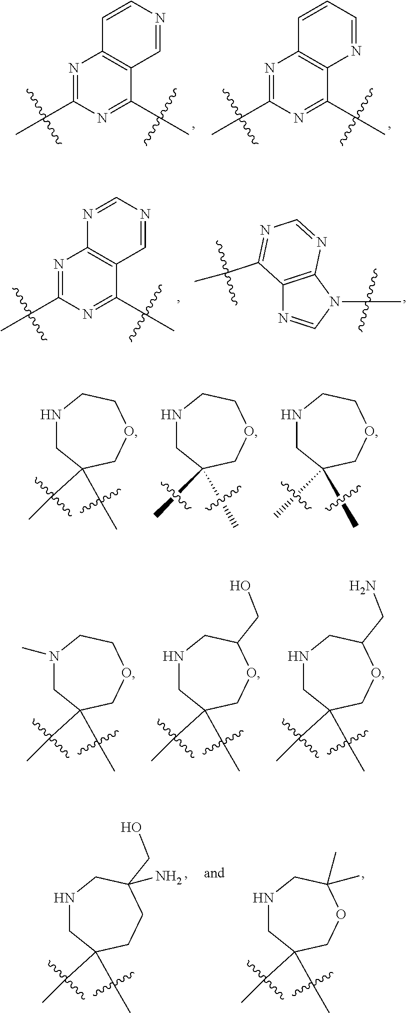

more particularly, A⁶ is selected from:

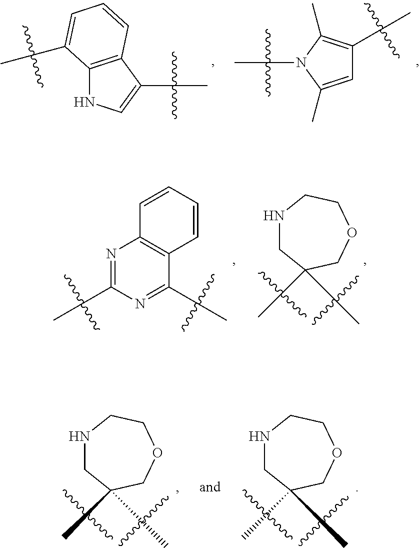

3. The MAP4K3 inhibitor of aspect 1 or aspect 2, wherein the MAP4K3 inhibitor comprises the structure of Formula I(a):

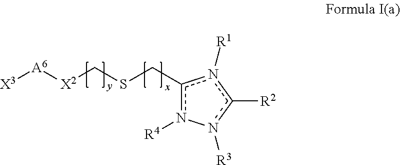

or a pharmaceutically acceptable salt, solvate, or prodrug thereof,
wherein,

A⁶ is a nitrogen containing optionally substituted heterocycle, wherein the nitrogen containing heterocycle can comprise one or more ring structures, including fused ring structures;

R¹-R⁴ are each individually absent or selected from H, D, optionally substituted (C₁-C₆)-alkyl, optionally substituted (C₁-C₆)-heteroalkyl, optionally substituted (C₂-C₆)-alkenyl, optionally substituted (C₂₋₆)-heteroalkenyl, optionally substituted (C₂₋₆)-alkynyl, optionally substituted (C₂-C₆)-heteroalkynyl, optionally substituted (C₃-C₈)-cycloalkyl, optionally substituted (C₄-C₈)-cycloalkenyl, optionally substituted aryl, optionally substituted heterocycle, halo, hydroxyl, ketone, aldehyde, acyl halide, carbonate, carboxylic acid, ester, ether, amide, amine, imine, azide, cyanate, azo, nitrate, nitrile, nitro, and nitroso, thiol, sulfide, disulfide, sulfoxide, sulfone, sulfinic acid, sulfonic acid, thiocyanate, thione, thial, phosphine, phosphonic acid, phosphate, phosphodiester, boronic acid, boronic ester, borinic acid, and borinic ester;

R¹³-R¹⁴ are each individually selected from H, D, halo, and an optionally substituted (C₁-C₆)-alkyl;

X² is NR¹³,

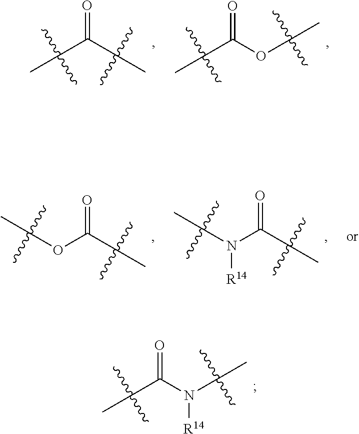

X³ is selected from H, D, halo, hydroxyl, amine, optionally substituted (C₁-C₆)-alkyl, azido, nitrile, and a (C₁-C₆)-alkylammonium group;

x is an integer selected from 0 or 1;

y is an integer selected from 1, 2, or 3; and

z is an integer selected from 0 or 1,

particularly, wherein the nitrogen containing optionally substituted heterocycle of A⁶ is either unsubstituted or substituted with one or more of the following groups: (C₁-C₆)alkyl, (C₁-C₆)-heteroalkyl, (C₂-C₆)-alkenyl, (C₂₋₆)-heteroalkenyl, aryl, heterocycle, halo, hydroxyl, aldehyde, carboxylic acid, ester, ether, amide, amine, nitro, and thiol, more particularly, wherein the nitrogen containing optionally substituted heterocycle of A⁶ is either unsubstituted or substituted with one or more of the following groups: (C₁-C₆)alkyl, halo, hydroxyl, carboxylic acid, amine, nitro, and thiol; and/or

particularly, R¹-R⁴ are each individually absent or selected from H, D, optionally substituted (C₁-C₆)-alkyl, optionally substituted (C₁-C₆)-heteroalkyl, optionally substituted (C₂-C₆)-alkenyl, optionally substituted (C₃-C₈)-cycloalkyl, optionally substituted aryl, optionally substituted heterocycle, halo, hydroxyl, carboxylic acid, ester, ether, amide, amine, azide, nitro, and thiol; and/or

particularly, X² is NR¹³ or

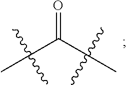 [SMILES: *C(*)=O]

and/or

particularly, X³ is selected from H, halo, hydroxyl, amine, and an optionally substituted (C₁-C₆)-alkyl; and/or
particularly, the MAP4K3 inhibitor does not have a structure selected from:

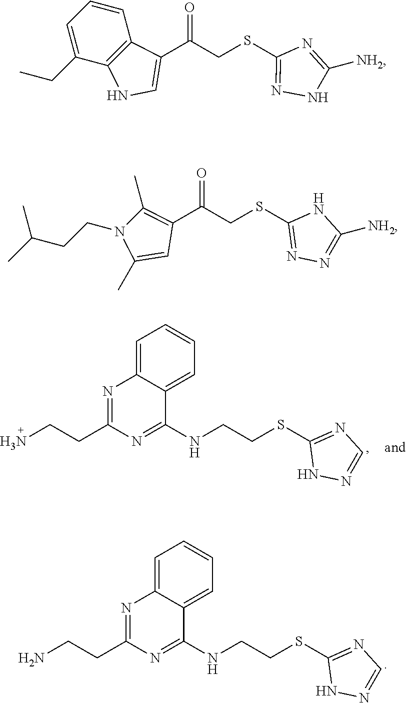 [SMILES: CCc1cccc2c(C(=O)CSc3n[nH]c(N)n3)c[nH]c12 ; Cc1cc(C(=O)CSc2nnc(N)[nH]2)c(C)n1CCC(C)C ; NCCc1nc(NCCSc2ncn[nH]2)c2ccccc2n1 ; NCCc1nc(NCCSc2ncn[nH]2)c2ccccc2n1]

4. The MAPK3 inhibitor of aspect 3, wherein A⁶ is selected from:

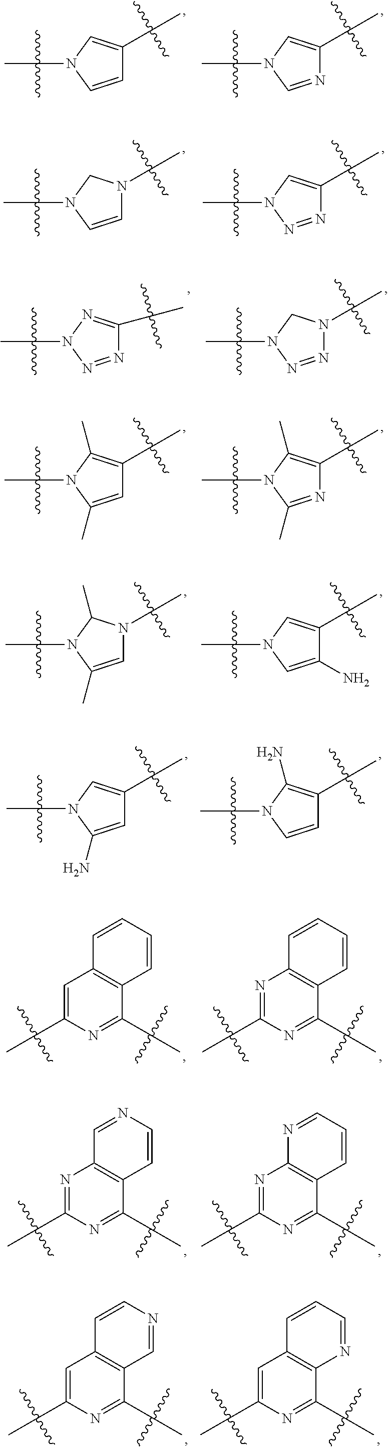

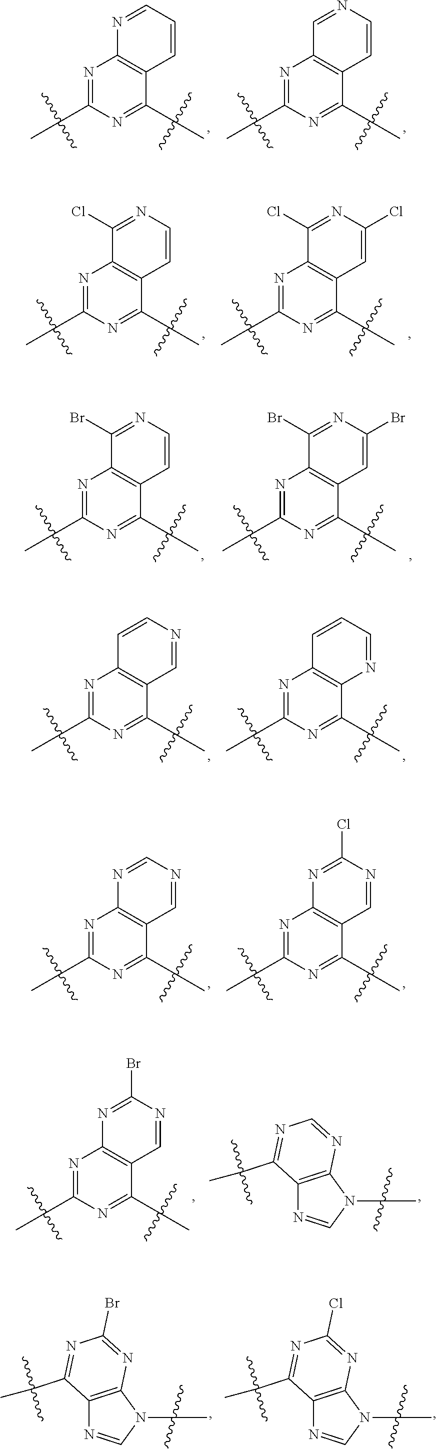

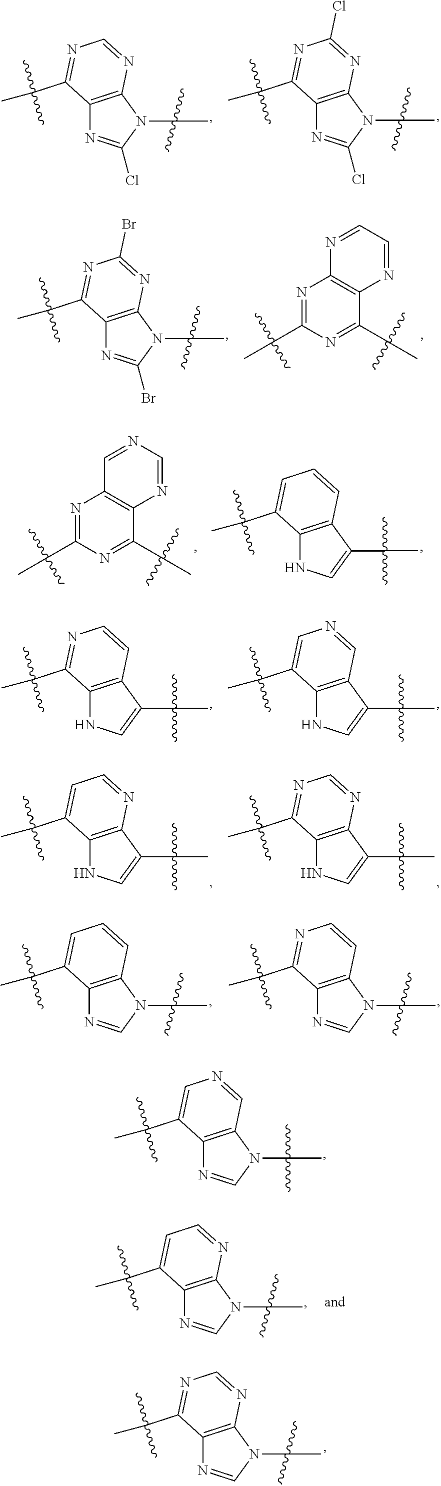

particularly, A⁶ is selected from:

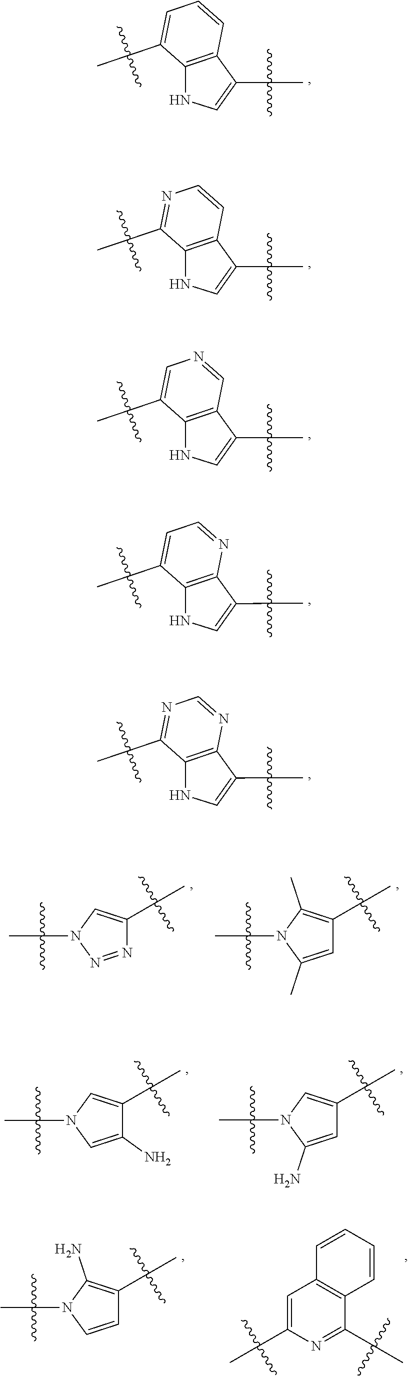

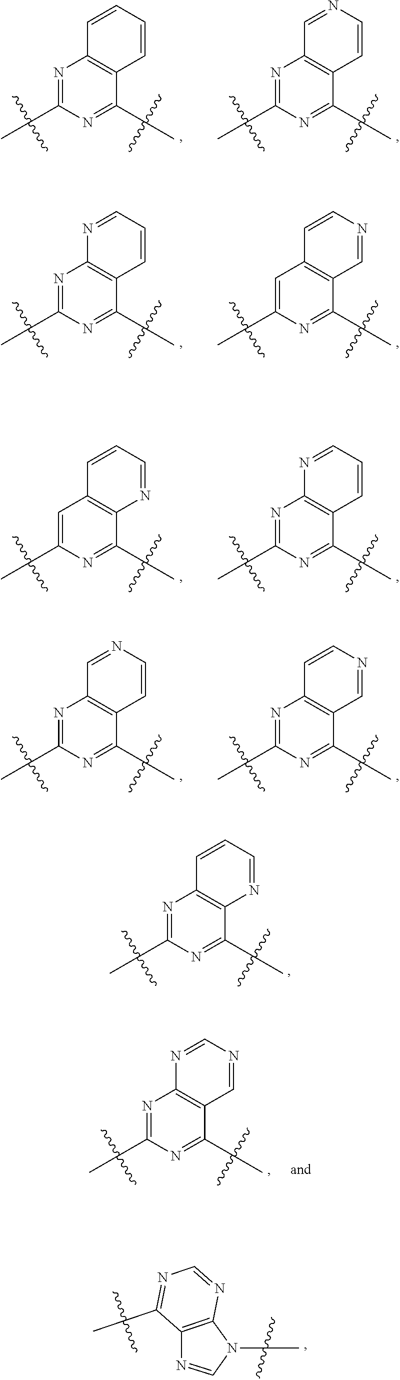

more particularly, A⁶ is selected from:

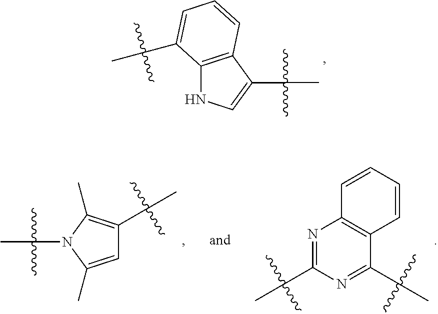

5. The MAP4K3 of any one of the previous aspects, wherein the MAP4K3 inhibitor comprises the structure of Formula I(b):

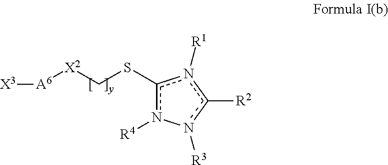

or a pharmaceutically acceptable salt, solvate, or prodrug thereof,
wherein,

A⁶ is selected from

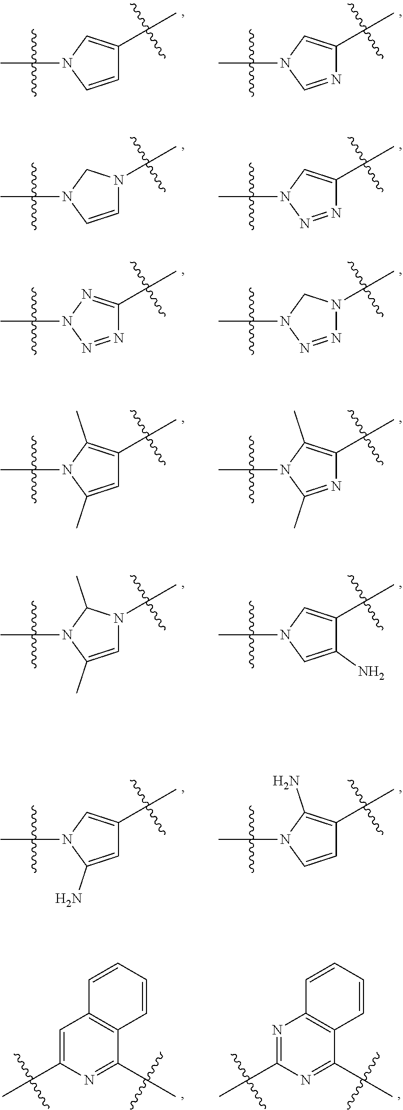

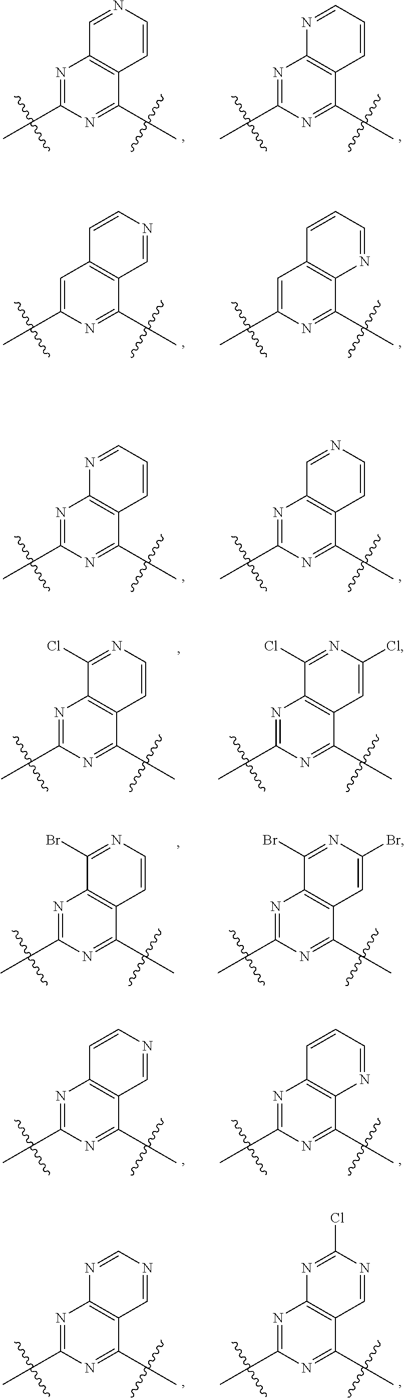

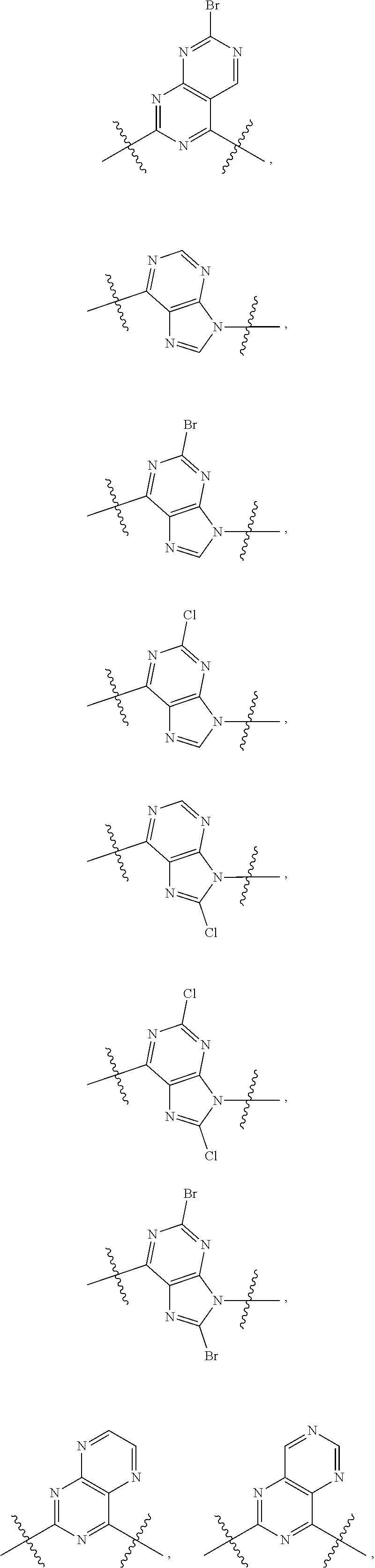

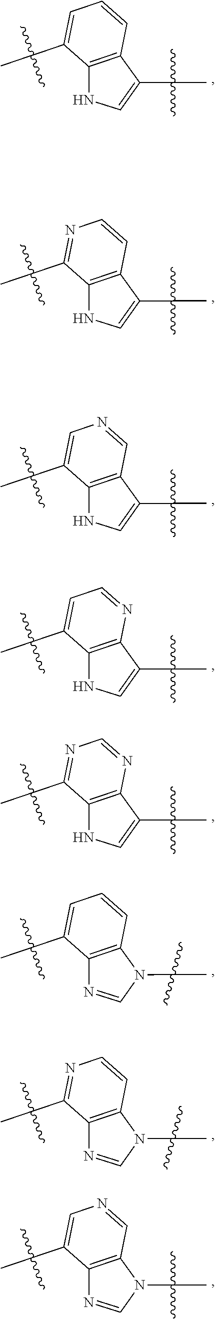

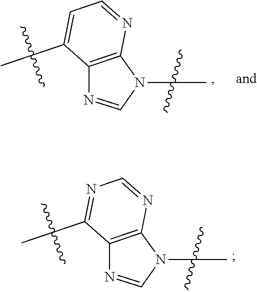 [SMILES: *c1ccnc2c1ncn2* ; *c1ncnc2c1ncn2*]

R¹-R⁴ are each individually absent or selected from H, D, optionally substituted (C₁-C₆)-alkyl, optionally substituted (C₁-C₆)-heteroalkyl, optionally substituted (C₂-C₆)-alkenyl, optionally substituted (C₂₋₆)-heteroalkenyl, optionally substituted (C₂₋₆)-alkynyl, optionally substituted (C₂-C₆)-heteroalkynyl, optionally substituted (C₃-C₈)-cycloalkyl, optionally substituted (C₄-C₈)-cycloalkenyl, optionally substituted aryl, optionally substituted heterocycle, halo, hydroxyl, ketone, aldehyde, acyl halide, carbonate, carboxylic acid, ester, ether, amide, amine, imine, azide, cyanate, azo, nitrate, nitrile, nitro, and nitroso, thiol, sulfide, disulfide, sulfoxide, sulfone, sulfinic acid, sulfonic acid, thiocyanate, thione, thial, phosphine, phosphonic acid, phosphate, phosphodiester, boronic acid, boronic ester, borinic acid, and borinic ester;

R¹³ is selected from H, D, halo, and an optionally substituted (C₁-C₆)-alkyl;

X² is NR¹³ or

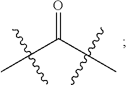 [SMILES: *C(*)=O]

X³ is an optionally substituted (C₁-C₆)-alkyl; and

y is an integer selected from 1, 2, or 3,

particularly, R¹-R⁴ are each individually absent or selected from H, D, optionally substituted (C₁-C₆)-alkyl, optionally substituted (C₁-C₆)-heteroalkyl, optionally substituted (C₂-C₆)-alkenyl, optionally substituted (C₃-C₈)-cycloalkyl, optionally substituted aryl, optionally substituted heterocycle, halo, hydroxyl, carboxylic acid, ester, ether, amide, amine, azide, nitro, and thiol; and/or

particularly, A⁶ is selected from:

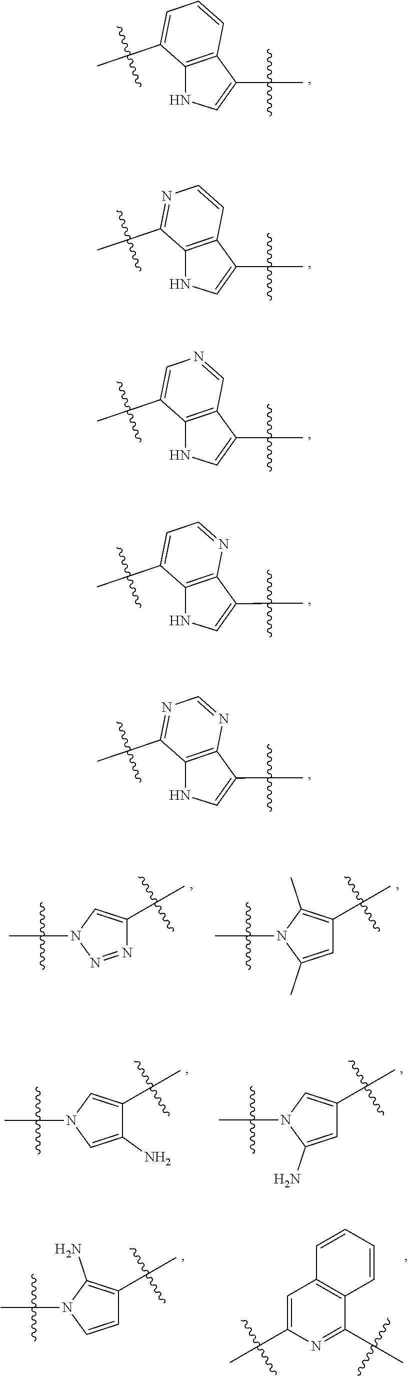

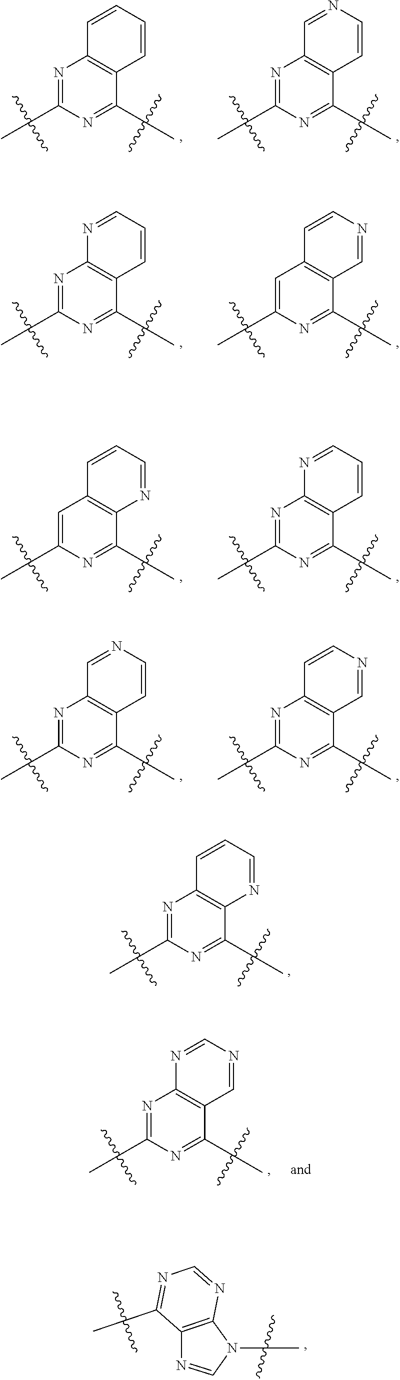

more particularly, A⁶ is selected from:

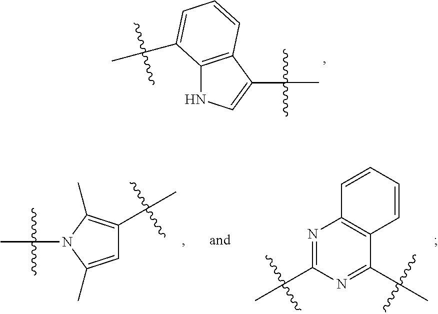

particularly, wherein the MAP4K3 inhibitor does not have the structure of

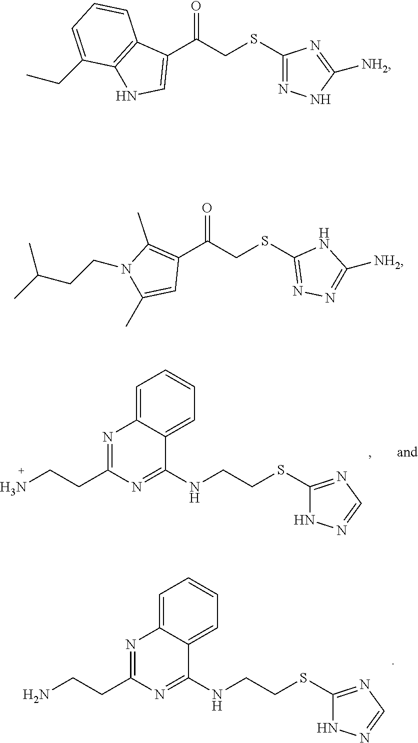 [SMILES: CCc1cccc2c(C(=O)CSc3n[nH]c(N)n3)c[nH]c12 ; Cc1cc(C(=O)CSc2nnc(N)[nH]2)c(C)n1CCC(C)C ; NCCc1nc(NCCSc2ncn[nH]2)c2ccccc2n1 ; NCCc1nc(NCCSc2ncn[nH]2)c2ccccc2n1]

6. The MAP4K3 of aspect 1, wherein the MAP4K3 inhibitor comprises the structure of Formula I(c):

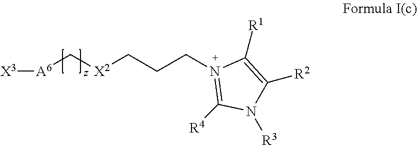

or a pharmaceutically acceptable salt, solvate, or prodrug thereof,
wherein,

A⁶ is selected from

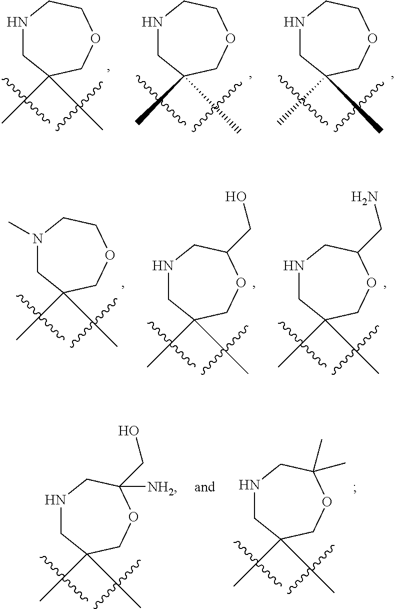

R¹³-R¹⁴ are each individually selected from H and an optionally substituted (C₁-C₆)-alkyl;

X² is selected from NR¹³,

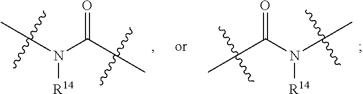

X³ is selected from H, D, halo, hydroxyl, amine, azido, and a nitrile; and

z is an integer selected from 1, 2, or 3,

particularly, A⁶ is selected from

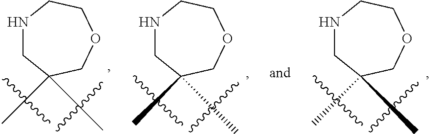

and and/or

particularly, X² is NR¹²; and/or

particularly, X³ is a hydroxyl or an amine; and/or

particularly, wherein the MAP4K3 inhibitor does not have the structure of:

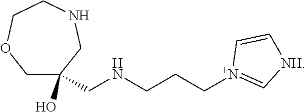 [SMILES: O[C@]1(CNCCC[n+]2cc[nH]c2)CNCCOC1]

7. The MAP4K3 inhibitor of any one of the previous aspects, wherein the MAP4K3 inhibitor inhibits MAP4K3 phosphorylation of PKCθThr538/total PKCθ by at least 40%,

particularly, wherein the MAP4K3 inhibitor inhibits MAP4K3 phosphorylation of PKCθThr538/total PKCθ by at least 60%,

more particularly, wherein the MAP4K3 inhibitor inhibits MAP4K3 phosphorylation of PKCθThr538/total PKCθ by at least 80%.

8. A pharmaceutical composition comprising the MAP4K3 inhibitor of any one of the previous aspects and a pharmaceutically acceptable excipient, diluent and/or carrier,

particularly, the pharmaceutical composition comprises a pharmaceutically acceptable salt of the MAP4K3 inhibitor.

9. The pharmaceutical composition of aspect 8, wherein the pharmaceutical composition is formulated for oral, parenteral, or topical delivery,

particularly, the pharmaceutical composition is formulated as a pill, tablet, or capsule for oral delivery.

10. A method of treating a MAP4K3-mediated disorder in a subject, comprising administering to the subject in need thereof a therapeutically effective amount of the MAP4K3 inhibitor of any one of aspects 1 to 7, or the pharmaceutical composition of aspect 8 or 9.

11. The method of aspect 10, wherein the MAP4K3-mediated disorder is a disorder associated with overexpression of MAP4K3, mis-regulation of MAP4K3, abnormal levels of MAP4K3, and/or abnormal activity of MAP4K3.

12. The method of aspect 10, wherein the MAP4K3-mediated disorder is a cancer, neurological disease, autoimmune disorder, or aging.

13. The method of aspect 12, wherein the cancer is selected from metastatic cancer, human renal cancer, lung cancer, liver cancer, pancreatic cancer, prostate cancer, endometrial cancer, melanoma, glioblastoma, and thyroid cancer,

particularly, the cancer is metastatic cancer, glioblastoma, or thyroid cancer.

14. The method of aspect 12, wherein the neurological disease is selected from tuberous sclerosis, epilepsy, Fragile X syndrome, Down syndrome, Rett syndrome, Alzheimer's disease, Parkinson's disease, and Huntington's disease,

particularly, the neurological disease is Fragile X syndrome, Alzheimer's disease, or Parkinson's disease.

15. The method of aspect 12, wherein the autoimmune disorder is selected from collagen induced arthritis (CIA), experimental autoimmune encephalomyelitis (EAE), inflammatory bowel disease (IBD), systemic lupus erythematosus (SLE), rheumatoid arthritis (RA), multiple sclerosis (MS), Type 1 diabetes mellitus, Guillain-Barre syndrome, psoriasis, chronic inflammatory demyelinating polyneuropathy, Graves' disease, Hashimoto's thyroiditis, myasthenia gravis, vasculitis, Sjögren syndrome, Addison disease, celiac disease, dermatomyositis, and adult-onset Still's disease (AOSD).

16. The method of any one of aspects 10 to 15, wherein the MAP4K3 inhibitor is administered in combination with one or more additional therapeutics or agents,

particularly, the one or more additional therapeutics or agents are anticancer agents and/or immunosuppressive agents.

17. A screening method to identify MAP4K3 inhibitors, comprising:

identifying compounds in an in vitro kinase assay which inhibit the phosphorylation of PKCθThr538/total PKCθ by at least 40%,

particularly, wherein the MAP4K3 inhibitor inhibits MAP4K3 phosphorylation of PKCθThr538/total PKCθ by at least 60%,

more particularly, wherein the MAP4K3 inhibitor inhibits MAP4K3 phosphorylation of PKCθThr538/total PKCθ by at least 80%.

18. The screening method of aspect 17, wherein the method further comprises one or more of the followings steps for the compounds that are found to inhibit the phosphorylation of PKCθThr538/total PKCθ by at least 40%:

identifying compounds that provide for at least 50% of TFEB in nuclei; and/or

identifying compounds that provide for at least 20% increase in LC3-II flux/LC3-I normalized to β-actin+/−bafilomycin; and/or

identifying compounds that provide for at least a 2.0-fold increase in the number of autolysosomes.

A number of embodiments have been described herein. Nevertheless, it will be understood that various modifications may be made without departing from the spirit and scope of this disclosure. Accordingly, other embodiments are within the scope of the following claims.

What is claimed is:

1. A MAP4K3 inhibitor comprising the structure of Formula I: 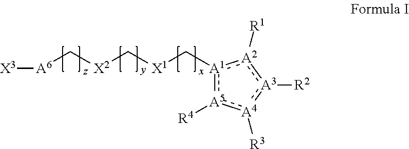
or a pharmaceutically acceptable salt, solvate, or prodrug thereof,
wherein,
A¹ is N⁺, C or CR⁵;
A² is N, C or CR⁶;
A³ is N, C or CR⁷;
A⁴ is N, C or CR⁸;
A⁵ is N, C or CR⁹;
A⁶ is a nitrogen containing optionally substituted heterocycle, wherein the nitrogen containing heterocycle can comprise one or more ring structures, including fused ring structures;
R¹-R⁴ are each individually absent or selected from H, D, optionally substituted (C₁-C₁₂)-alkyl, optionally substituted (C₁-C₁₂)-heteroalkyl, optionally substituted (C₂-C₁₂)-alkenyl, optionally substituted (C₂₋₁₂)-heteroalkenyl, optionally substituted (C₂₋₁₂)-alkynyl, optionally substituted (C₂-C₁₂)-heteroalkynyl, optionally substituted (C₃-C₁₂)-cycloalkyl, optionally substituted (C₄-C₁₂)-cycloalkenyl, optionally substituted aryl, optionally substituted heterocycle, halo, hydroxyl, ketone, aldehyde, acyl halide, carbonate, carboxylic acid, ester, ether, amide, amine, imine, azide, cyanate, azo, nitrate, nitrile, nitro, nitroso, thiol, sulfide, disulfide, sulfoxide, sulfone, sulfinic acid, sulfonic acid, thiocyanate, thione, thial, phosphine, phosphonic acid, phosphate, phosphodiester, boronic acid, boronic ester, borinic acid, and borinic ester;
R⁵-R⁹ are each individually selected from H, D, optionally substituted (C₁-C₁₂)-alkyl, optionally substituted (C₁-C₁₂)-heteroalkyl, optionally substituted (C₂-C₁₂)-alkenyl, optionally substituted (C₂₋₁₂)-heteroalkenyl, optionally substituted (C₂₋₁₂)-alkynyl, optionally substituted (C₂-C₁₂)-heteroalkynyl, optionally substituted (C₃-C₁₂)-cycloalkyl, optionally substituted (C₄-C₁₂)-cycloalkenyl, optionally substituted aryl, optionally substituted heterocycle, halo, hydroxyl, ketone, aldehyde, acyl halide, carbonate, carboxylic acid, ester, ether, amide, amine, imine, azide, cyanate, azo, nitrate, nitrile, nitro, nitroso, thiol, sulfide, disulfide, sulfoxide, sulfone, sulfinic acid, sulfonic acid, thiocyanate, thione, thial, phosphine, phosphonic acid, phosphate, phosphodiester, boronic acid, boronic ester, borinic acid, and borinic ester;
R¹⁰-R¹⁴ are each individually selected from H, D, halo, and an optionally substituted (C₁-C₆)-alkyl;
X¹ is S, O, CR¹⁰R¹¹ or NR¹²;
X² is NR¹³, 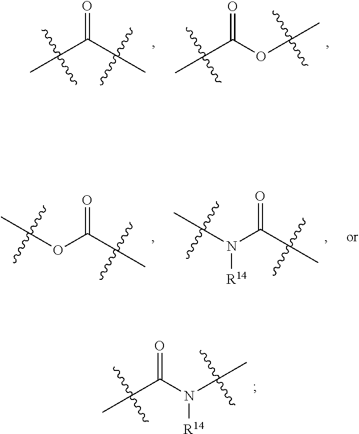
X³ is selected from H, D, halo, hydroxyl, amine, optionally substituted (C₁-C₆)-alkyl, azido, nitrile, and a (C₁-C₆)-alkylammonium group;
x is an integer selected from 0, 1, 2, or 3;
y is an integer selected from 0, 1, 2 or 3;
z is an integer selected from 0, 1, 2, or 3;
wherein, at least 2 of A¹-A⁵ comprise nitrogen containing groups; wherein, there is only one double bond located between one of the following recited groups: A¹ and A⁵, and A¹ and A²;
wherein there is only one double bond located between one of the following recited groups: A² and A³, A³ and A⁴, and A⁴ and A⁵; and
with the proviso that the MAP4K3 inhibitor does not have a structure selected from:

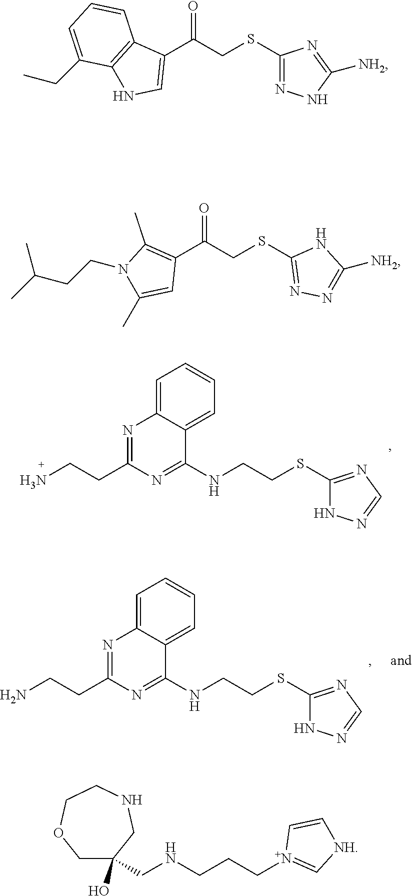 [SMILES: CCc1cccc2c(C(=O)CSc3n[nH]c(N)n3)c[nH]c12 ; Cc1cc(C(=O)CSc2nnc(N)[nH]2)c(C)n1CCC(C)C ; NCCc1nc(NCCSc2ncn[nH]2)c2ccccc2n1 ; NCCc1nc(NCCSc2ncn[nH]2)c2ccccc2n1 ; O[C@]1(CNCCC[n+]2cc[nH]c2)CNCCOC1]

2. The MAP4K3 inhibitor of claim 1, wherein A⁶ is selected from:

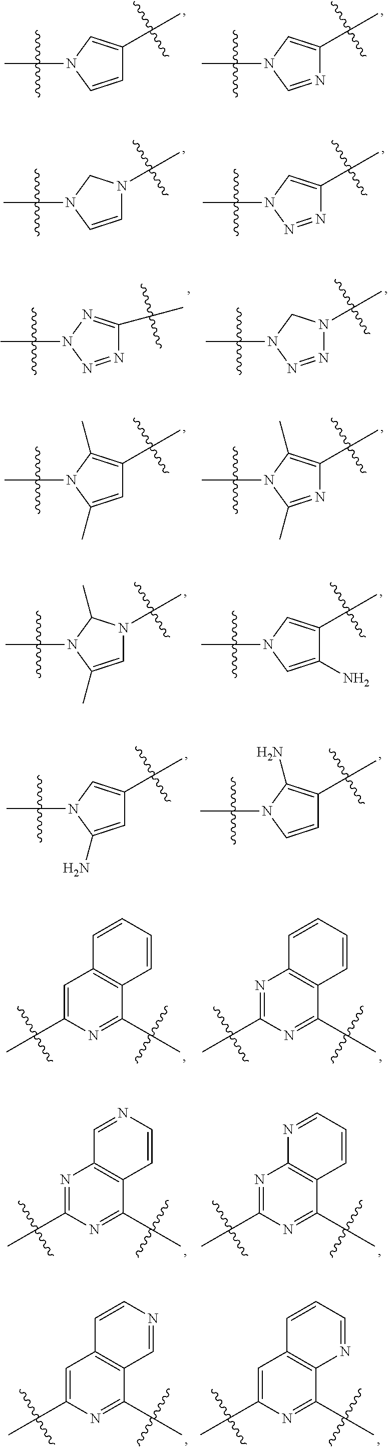

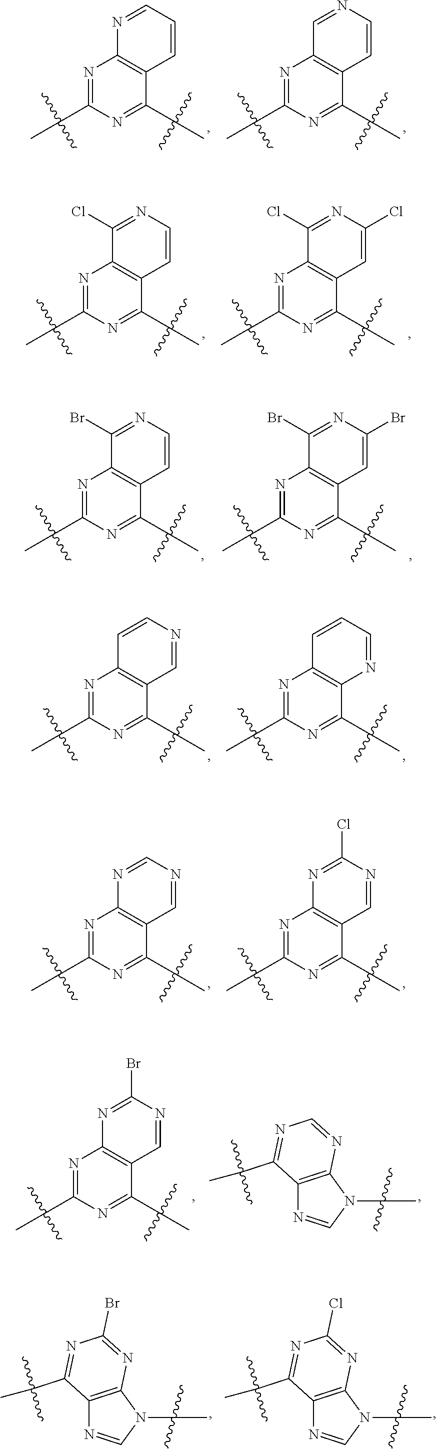

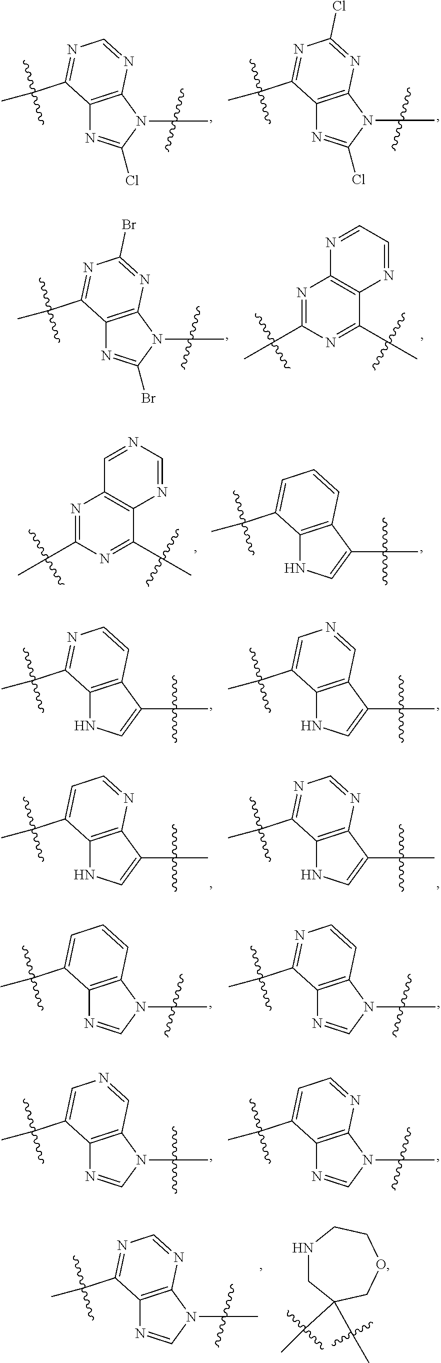

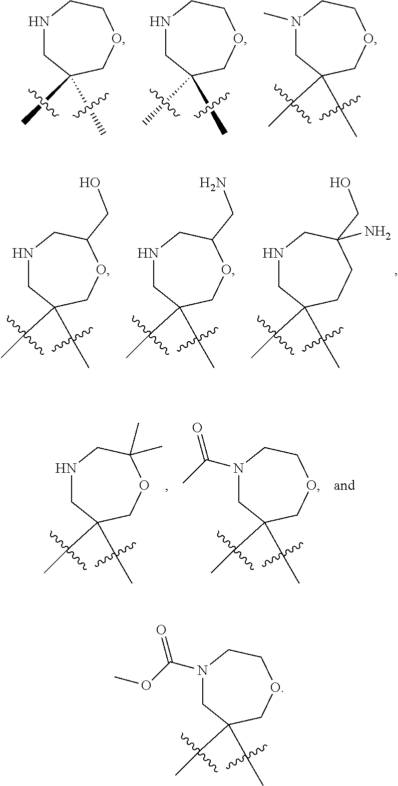

3. The MAP4K3 inhibitor of claim 1, wherein the MAP4K3 inhibitor comprises the structure of Formula I(b): 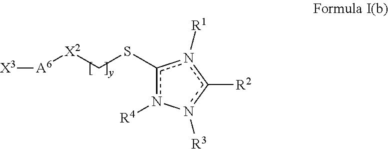
or a pharmaceutically acceptable salt, solvate, or prodrug thereof,
wherein
A⁶ is selected from 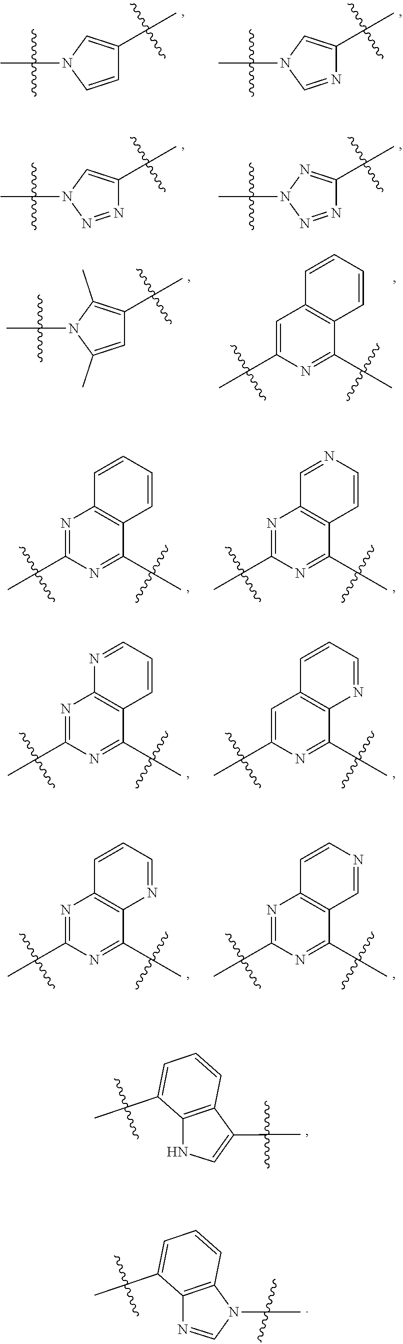 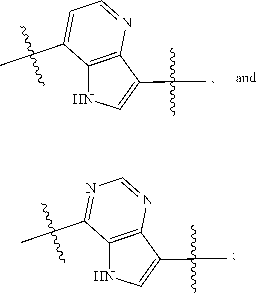 [SMILES: *c1c[nH]c2c(*)ccnc12 ; *c1c[nH]c2c(*)ncnc12]
R¹-R⁴ are each individually absent or selected from H, D, optionally substituted (C₁-C₆)-alkyl, optionally substituted (C₁-C₆)-heteroalkyl, optionally substituted (C₂-C₆)-alkenyl, optionally substituted (C₂₋₆)-heteroalkenyl, optionally substituted (C₂₋₆)-alkynyl, optionally substituted (C₂-C₆)-heteroalkynyl, optionally substituted (C₃-C₈)-cycloalkyl, optionally substituted (C₄-C₈)-cycloalkenyl, optionally substituted aryl, optionally substituted heterocycle, halo, hydroxyl, ketone, aldehyde, acyl halide, carbonate, carboxylic acid, ester, ether, amide, amine, imine, azide, cyanate, azo, nitrate, nitrile, nitro, and nitroso, thiol, sulfide, disulfide, sulfoxide, sulfone, sulfinic acid, sulfonic acid, thiocyanate, thione, thial, phosphine, phosphonic acid, phosphate, phosphodiester, boronic acid, boronic ester, borinic acid, and borinic ester;
R¹³ is selected from H, D, halo, and an optionally substituted (C₁-C₆)-alkyl;
X² is NR¹³ or 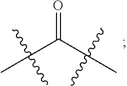 [SMILES: *C(*)=O]
X³ is an optionally substituted (C₁-C₆)-alkyl group;
y is an integer selected from 1, 2, or 3; and
with the proviso that the MAP4K3 inhibitor does not have a structure selected from:

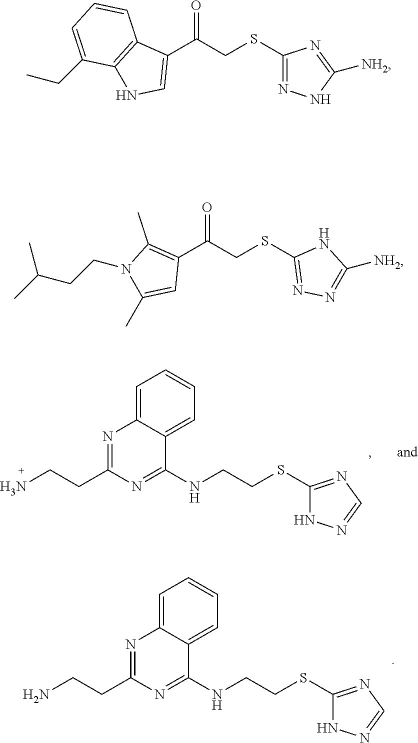 [SMILES: CCc1cccc2c(C(=O)CSc3n[nH]c(N)n3)c[nH]c12 ; Cc1cc(C(=O)CSc2nnc(N)[nH]2)c(C)n1CCC(C)C ; NCCc1nc(NCCSc2ncn[nH]2)c2ccccc2n1 ; NCCc1nc(NCCSc2ncn[nH]2)c2ccccc2n1]

4. The MAP4K3 of claim 1, wherein the MAP4K3 inhibitor comprises the structure of Formula I(c): 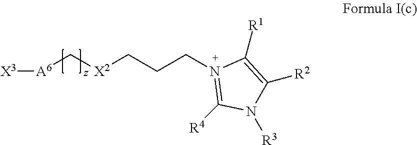
or a pharmaceutically acceptable salt, solvate, or prodrug thereof,
wherein,
A⁶ is selected from 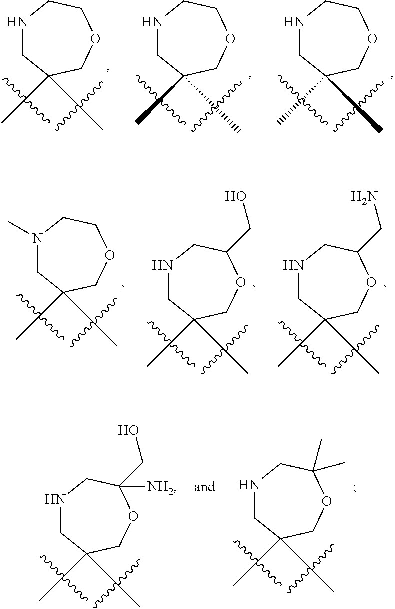
R¹³-R¹⁴ are each individually selected from H and an optionally substituted (C₁-C₆)-alkyl;
X² is selected from NR¹³, 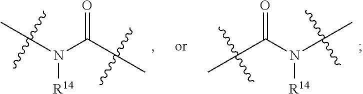
X³ is selected from H, D, halo, hydroxyl, amine, azido, and a nitrile;
z is an integer selected from 2, or 3; and
with the proviso that the MAP4K3 inhibitor does not have the structure of:

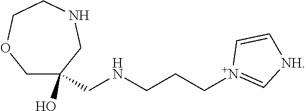 [SMILES: O[C@]1(CNCCC[n+]2cc[nH]c2)CNCCOC1]

5. The MAP4K3 inhibitor of claim 1, wherein the MAP4K3 inhibitor inhibits MAP4K3 phosphorylation of PKCθThr538/total PKCθ by at least 40%.

6. A pharmaceutical composition comprising the MAP4K3 inhibitor of claim 1 and a pharmaceutically acceptable excipient, diluent and/or carrier.

7. The pharmaceutical composition of claim 6, wherein the pharmaceutical composition is formulated for oral, parenteral, or topical delivery.

8. A method of treating a MAP4K3-mediated disorder in a subject, comprising administering to the subject in need thereof a therapeutically effective amount of the MAP4K3 inhibitor comprising the structure of Formula I: 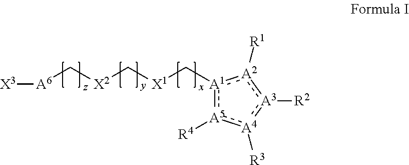
or a pharmaceutically acceptable salt, solvate, or prodrug thereof,
wherein,
A¹ is N⁺, C or CR⁵;
A² is N, C or CR⁶;
A³ is N, C or CR⁷;
A⁴ is N, C or CR⁸;
A⁵ is N, C or CR⁹;
A⁶ is a nitrogen containing optionally substituted heterocycle, wherein the nitrogen containing heterocycle can comprise one or more ring structures, including fused ring structures;
R¹-R⁴ are each individually absent or selected from H, D, optionally substituted (C₁-C₁₂)-alkyl, optionally substituted (C₁-C₁₂)-heteroalkyl, optionally substituted (C₂-C₁₂)-alkenyl, optionally substituted (C₂₋₁₂)-heteroalkenyl, optionally substituted (C₂₋₁₂)-alkynyl, optionally substituted (C₂-C₁₂)-heteroalkynyl, optionally substituted (C₃-C₁₂)-cycloalkyl, optionally substituted (C₄-C₁₂)-cycloalkenyl, optionally substituted aryl, optionally substituted heterocycle, halo, hydroxyl, ketone, aldehyde, acyl halide, carbonate, carboxylic acid, ester, ether, amide, amine, imine, azide, cyanate, azo, nitrate, nitrile, nitro, nitroso, thiol, sulfide, disulfide, sulfoxide, sulfone, sulfinic acid, sulfonic acid, thiocyanate, thione, thial, phosphine, phosphonic acid, phosphate, phosphodiester, boronic acid, boronic ester, borinic acid, and borinic ester;
R⁵-R⁹ are each individually selected from H, D, optionally substituted (C₁-C₁₂)-alkyl, optionally substituted (C₁-C₁₂)-heteroalkyl, optionally substituted (C₂-C₁₂)-alkenyl, optionally substituted (C₂₋₁₂)-heteroalkenyl, optionally substituted (C₂₋₁₂)-alkynyl, optionally substituted (C₂-C₁₂)-heteroalkynyl, optionally substituted (C₃-C₁₂)-cycloalkyl, optionally substituted (C₄-C₁₂)-cycloalkenyl, optionally substituted aryl, optionally substituted heterocycle, halo, hydroxyl, ketone, aldehyde, acyl halide, carbonate, carboxylic acid, ester, ether, amide, amine, imine, azide, cyanate, azo, nitrate, nitrile, nitro, nitroso, thiol, sulfide, disulfide, sulfoxide, sulfone, sulfinic acid, sulfonic acid, thiocyanate, thione, thial, phosphine, phosphonic acid, phosphate, phosphodiester, boronic acid, boronic ester, borinic acid, and borinic ester;
R¹⁰-R¹⁴ are each individually selected from H, D, halo, and an optionally substituted (C₁-C₆)-alkyl;
X¹ is S, O, CR¹⁰R¹¹ or NR¹²;
X² is NR¹³, 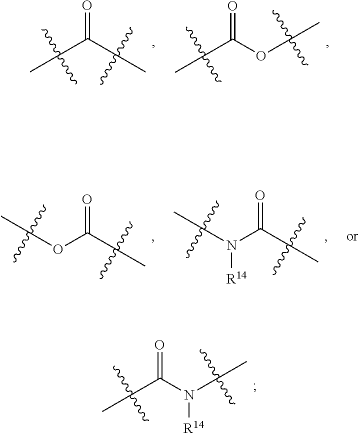
X³ is selected from H, D, halo, hydroxyl, amine, optionally substituted (C₁-C₆)-alkyl, azido, nitrile, and a (C₁-C₆)-alkylammonium group;
x is an integer selected from 0, 1, 2, or 3;
y is an integer selected from 0, 1, 2 or 3;
z is an integer selected from 0, 1, 2, or 3;
wherein, at least 2 of A¹-A⁵ comprise nitrogen containing groups, wherein, there is only one double bond located between one of the following recited groups: A¹ and A⁵, and A¹ and A²;
and wherein, there is only one double bond located between one of the following recited groups: A² and A³, A³ and A⁴, and A⁴ and A⁵.

9. The method of claim 8, wherein A⁶ is selected from:

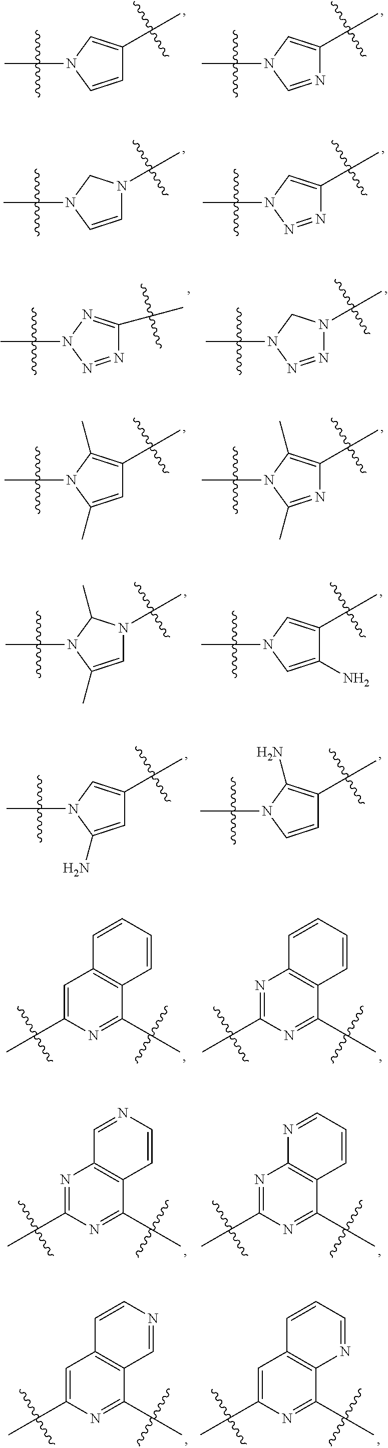

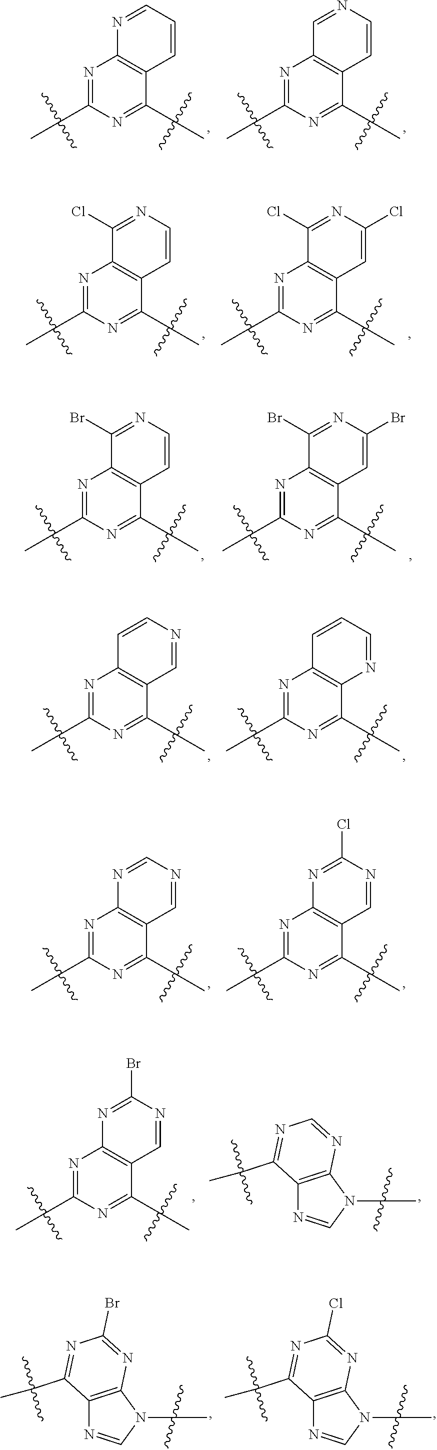

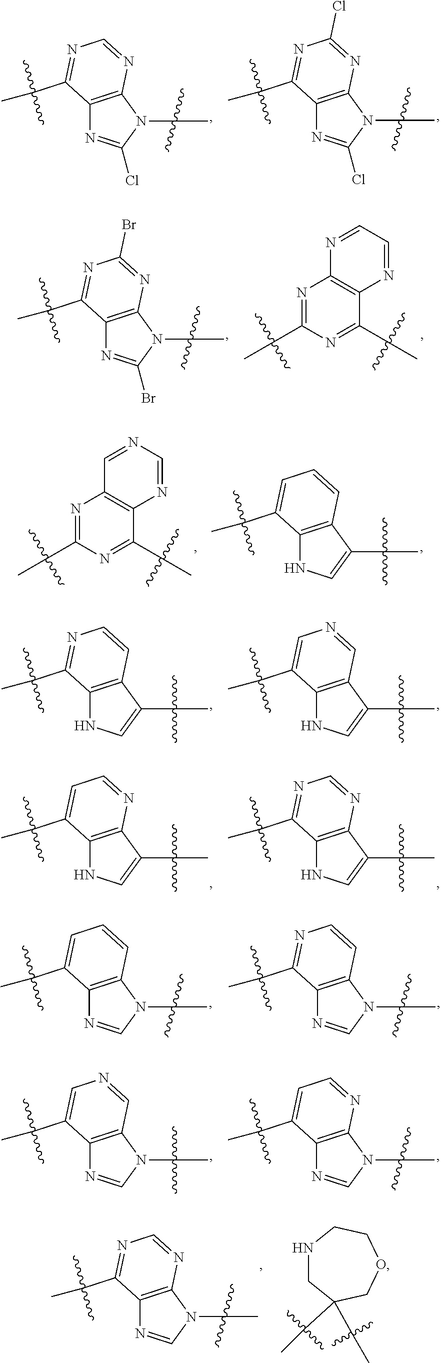

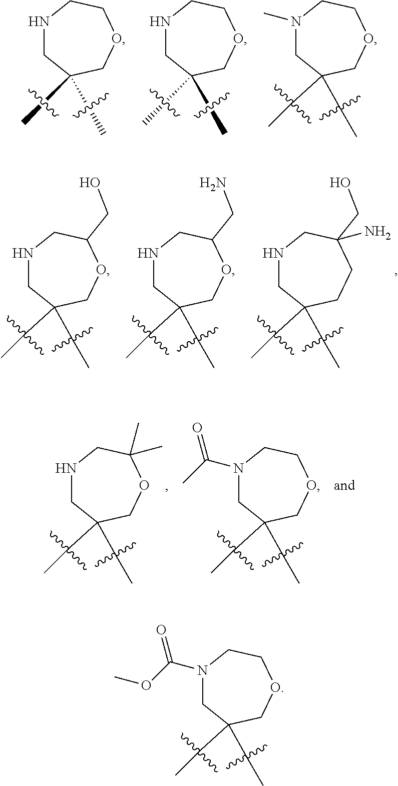

10. The method of claim 8, wherein the MAP4K3 inhibitor comprises the structure of Formula I(b): 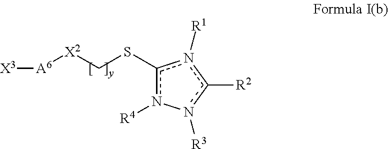
or a pharmaceutically acceptable salt, solvate, or prodrug thereof,
wherein,
A⁶ is selected from 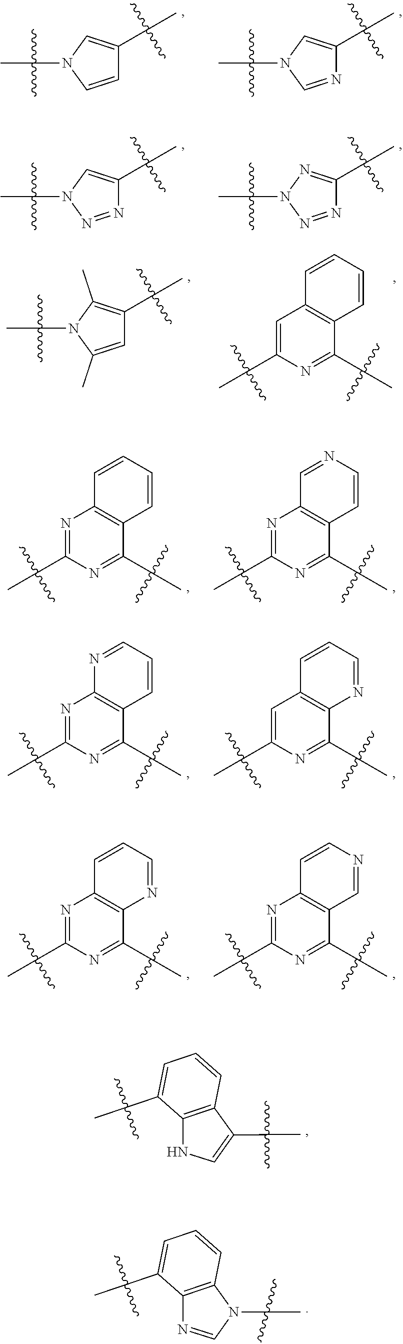 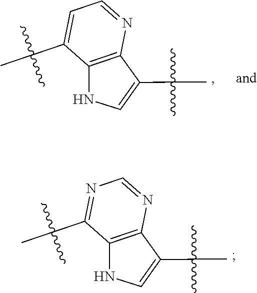 [SMILES: *c1c[nH]c2c(*)ccnc12 ; *c1c[nH]c2c(*)ncnc12]
R¹-R⁴ are each individually absent or selected from H, D, optionally substituted (C₁-C₆)-alkyl, optionally substituted (C₁-C₆)-heteroalkyl, optionally substituted (C₂-C₆)-alkenyl, optionally substituted (C₂₋₆)-heteroalkenyl, optionally substituted (C₂₋₆)-alkynyl, optionally substituted (C₂-C₆)-heteroalkynyl, optionally substituted (C₃-C₈)-cycloalkyl, optionally substituted (C₄-C₈)-cycloalkenyl, optionally substituted aryl, optionally substituted heterocycle, halo, hydroxyl, ketone, aldehyde, acyl halide, carbonate, carboxylic acid, ester, ether, amide, amine, imine, azide, cyanate, azo, nitrate, nitrile, nitro, and nitroso, thiol, sulfide, disulfide, sulfoxide, sulfone, sulfinic acid, sulfonic acid, thiocyanate, thione, thial, phosphine, phosphonic acid, phosphate, phosphodiester, boronic acid, boronic ester, borinic acid, and borinic ester;
R¹³ is selected from H, D, halo, and an optionally substituted (C₁-C₆)-alkyl;
X² is NR¹³ or 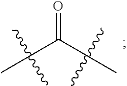 [SMILES: *C(*)=O]
X³ is an optionally substituted (C₁-C₆)-alkyl group;
y is an integer selected from 1, 2, or 3.

11. The method of claim 8, wherein the MAP4K3 inhibitor comprises the structure of Formula I(c): 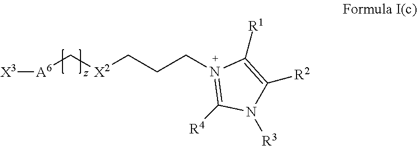
or a pharmaceutically acceptable salt, solvate, or prodrug thereof,
wherein,
A⁶ is selected from 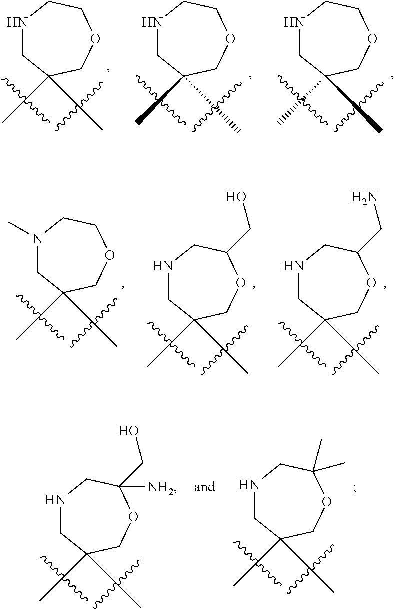
R¹³-R¹⁴ are each individually selected from H and an optionally substituted (C₁-C₆)-alkyl;
X² is selected from NR¹³, 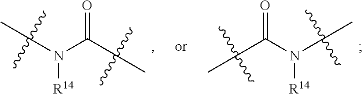
X³ is selected from H, D, halo, hydroxyl, amine, azido, and a nitrile;
z is an integer selected from 2, or 3.

12. The method of claim 8, wherein the MAP4K3 inhibitor has a structure selected from (a), (b), (c), (d) and (e): 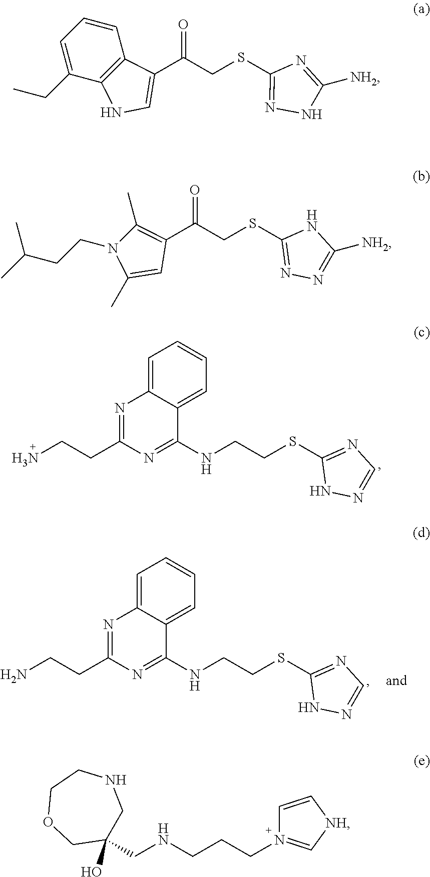
or a pharmaceutically acceptable salt, solvate, or prodrug thereof.

13. The method of claim 8, wherein the MAP4K3-mediated disorder is a disorder associated with overexpression of MAP4K3, mis-regulation of MAP4K3, abnormal levels of MAP4K3, and/or abnormal activity of MAP4K3.

14. The method of claim 8, wherein the MAP4K3-mediated disorder is a cancer, neurological disease, autoimmune disorder, or aging.

15. The method of claim 14, wherein the cancer is selected from metastatic cancer, human renal cancer, lung cancer, liver cancer, pancreatic cancer, prostate cancer, endometrial cancer, melanoma, glioblastoma, and thyroid cancer.

16. The method of claim 14, wherein the neurological disease is selected from tuberous sclerosis, epilepsy, Fragile X syndrome, Down syndrome, Rett syndrome, Alzheimer's disease, Parkinson's disease, and Huntington's disease.

17. The method of claim 14, wherein the autoimmune disorder is selected from collagen induced arthritis (CIA), experimental autoimmune encephalomyelitis (EAE), inflammatory bowel disease (IBD), systemic lupus erythematosus (SLE), rheumatoid arthritis (RA), multiple sclerosis (MS), Type 1 diabetes mellitus, Guillain-Barre syndrome, psoriasis, chronic inflammatory demyelinating polyneuropathy, Graves' disease, Hashimoto's thyroiditis, myasthenia gravis, vasculitis, Sjögren syndrome, Addison disease, celiac disease, dermatomyositis, and adult-onset Still's disease (AOSD).

18. The method of claim 8, wherein the MAP4K3 inhibitor is administered in combination with one or more additional therapeutics or agents.

19. A screening method to identify MAP4K3 inhibitors, comprising:
identifying compounds in an in vitro kinase assay which inhibit the phosphorylation of PKCθThr538/total PKCθ by at least 40%.

20. The screening method of claim 19, wherein the method further comprises one or more of the followings steps for the compounds that are found to inhibit the phosphorylation of PKCθThr538/total PKCθ by at least 40%:
identifying compounds that provide for at least 50% of TFEB in nuclei; and/or
identifying compounds that provide for at least 20% increase in LC3-II flux/LC3-I normalized to β-actin+/−bafilomycin; and/or
identifying compounds that provide for at least a 2.0-fold increase in the number of autolysosomes.
